(19) United States
(12) Patent Application Publication
(10) Pub. No.: US 2008/0311135 A1
(43) Pub. Date: Dec. 18, 2008

(54) IMMUNE COMPLEX VACCINATION AS A STRATEGY TO ENHANCE IMMUNITY IN THE ELDERLY AND OTHER IMMUNE COMPROMISED POPULATIONS

(75) Inventors: Biao Zheng, Sugar Land, TX (US); Shuhua Han, Sugar Land, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE

(21) Appl. No.: 12/125,203

(22) Filed: May 22, 2008

Related U.S. Application Data
(60) Provisional application No. 60/939,541, filed on May 22, 2007.

(51) Int. Cl. A61K 39/395; A61P 37/02

(52) U.S. Cl. 424/178.1

(57) ABSTRACT

The present invention generally concerns methods and compositions for improving the immune system of an individual that is an immune-compromised individual. In particular aspects, the immune-compromised individual is elderly or is immunosuppressed, such as from chemotherapy or immunosuppressants following organ or tissue transplantation. In specific embodiments, the invention relates to delivery to the immune-compromised individual of immune complexes harboring an antigen and an antibody that immunologically recognizes the antigen. The antigen may be viral, bacterial, or fungal, for example.

### CROSS-REFERENCE TO RELATED APPLICATIONS

This application claims priority to U.S. Provisional Application No. 60/939,541, filed on May 22, 2007, and incorporated herein by reference in its entirety.

### STATEMENT REGARDING FEDERALLY SPONSORED RESEARCH OR DEVELOPMENT

The present invention was developed at least in part with funds from the National Institutes of Health Grants No. R01 AG17149 and A1062917. The United States Government has certain rights in the invention.

### FIELD OF THE INVENTION

The present invention generally concerns at least the fields of immunology, cell biology, and medicine. In particular aspects, the field of the invention relates to methods and compositions to enhance immunity in immune-compromised individuals.

### BACKGROUND OF THE INVENTION

After infection or immunization, aged individuals often generate significantly less antibodies (Miller, 1991), maintain protective titers of serum antibodies for much shorter periods (Kishimoto et al, 1980) and produce antibodies with affinity lower than that of young controls (Zharhary et al., 1977; Doria et al., 1978). These deficits are likely to be responsible for increased susceptibility to infection in aged populations. Germinal centers (GCs) are the principal sites of V(D)J somatic hypermutation (SHM), affinity-driven clonal selection, and generation of the memory and long-lived antibody-forming cell (AFC) compartments (McHeyzer-Williams et al., 1993; Kelsoe, 1995; MacLennan, 1994; Liu and Arpin, 1997; Jacob et al., 1991; Berek et al., 1991; Han et al., 1995). It has been shown that during memory responses, GC B cells from aged mice mutated their Ig genes at rates comparable to that of young mice (Han et al., 1995). In addition, Ag-driven clonal selection and affinity maturation are largely intact in aged mice. One of the major mechanisms responsible for aged-associated immune dysfunction is the impaired GC pathway of B cell differentiation and maturation, including T-dependent antibody (Ab) responses. This deficiency in GC reaction in aging leads to diminished antibody affinity maturation, poor memory response, and reduced long-term plasma cells in the bone marrow (Kosco et al., 1989; Zheng et al., 1997; Han et al., 2003; Miller and Kelsoe, 1995; Lu and Cerny, 2002). However, it has been shown that the intrinsic capability of aged B cells to be activated by the initial antigen stimulation is largely intact (Han et al., 2003; Han et al., 2004). On the other hand, in primary antibody responses to protein-based antigens, T cell help for B cell activation and differentiation is a major limiting factor (Maclennan et al., 1992). This limited T help becomes even more profound in old animals (Yang et al., 1996). It has been shown that the follicular B cell response was significantly more robust in mice whose T cells were primed with carrier proteins (Toellner et al., 1996). It is of great importance to identify means to overcome the age-associated GC dysfunction by bypassing the B cell requirement for signals from other components of the immune system, such as Th cells.

Fc receptors (FcRs) link the innate and adaptive branches of the immune system and have important functions in the activation and modulation of immune responses. Since both effector cells such as B cell and mast cells, as well as antigen-presenting cells (APCs), such as dendritic cells (DCs), follicular dendritic cells (FDCs) and B cells, express various types of FcRs, immune complex (IC) can exert their immunoregulatory functions by direct signaling effector cells and/or by targeting APCs. Thus, the advantages of IC in inducing immune responses are several-fold: ICs can directly activate effector cells; ICs are effectively taken up by professional APCs; and IC-binding to FcR can act as a natural adjuvant and mediate DC maturation. During an immune response, a small amount of IC is trapped on the cell processes of follicular dendritic cells (FDC) in the lymphoid follicles and retained for a long period (Tew et al., 1980; Nossal et al., 1968; Klaus and Humphrey, 1986), which plays a pivotal role in developing an effective T-dependent antibody response. ICs can stimulate B cells directly and lower the threshold of requirement for T cell help. The B-cell receptor (BCR) affinity threshold for antigen-uptake and presentation is significantly lowered by oligomerization of antigen with antibody (Tsoko et al., 1990; Carter and Fearon, 1992). ICs increase the avidity of antigen-BCR interaction and enhance the BCR-mediated signals. By fixing complement and bridging BCR with complement, ICs elicit costimulatory signals through co-receptors such as CD19 (Carter and Fearon, 1992; Dempsey et al., 1996). The co-ligation of BCR and complement receptors (CR) lowers the B-cell activation threshold by 100˜1,000-folds (Dempsey et al., 1996). In addition, capture of ICs by DCs contributes to an effective T cell response. IC binding leads to many other immunoregulatory and inflammatory responses, including phagocytosis, antibody-dependent cell-mediated cytotoxicity (ADCC), and release of cytokines and other inflammatory mediators central to the protective properties of antibodies (Ravetch and Clynes, 1998). It has been shown that engagement of activating FcγR induces DC activation and maturation (Regnault et al., 1999; Schuurhuis et al., 2002). FcγR-mediated internalization of ICs by DCs is associated with enhanced presentation of both MHC class I- and II-binding peptides derived from the antigens in the ICs (Regnault et al., 1999; Amigorena and Bonnerot, 1999a; 1999b). The contribution of FcγR and IC to both CD4⁺ Th cell (Hamano et al., 2000) and CD8+ CTL (Schuurhuis et al., 2002) functions has been demonstrated. These findings underscore the relevance of cross talk through DCs between the B and T cell compartments. The utilization of ICs to enhance the immune responses has been proved effective in inducing tumor immunity (Rafiq et al., 2002) and therapeutic vaccination for chronic viral infections such as hepatitis B (Zheng et al., 2001; Wen et al., 1999).

In the elderly, the state of dysregulated immune functions, or immunosenescence, compromises protection against infectious agents and contributes to the increased susceptibility to infectious diseases. Infectious diseases are major causes of morbidity and mortality in the elderly, accounting for about one third of all deaths in people 65 years of age or older (Mouton et al., 2001; Ginaldi et al., 2001; Gavazzi and Krause, 2002). In addition, there is a significant decrease in responsiveness to vaccination in aging. An impaired response to influenza infection and vaccination in the elderly may be the clinically most relevant fact associated with infectious diseases in aging (Thompson et al., 2003; Castel, 2000; Ginaldi et al., 1999). About 90% of as many as 50- to 70 thousands annual excess deaths attributed to influenza occur in people aged 65 years or older (Thompson et al., 2003; Castel, 2000). Even when the antigenic match between influenza vaccine and circulating virus is close, vaccination provides protection for only 30%-40% of subjects aged ≧65 years, compared with 70-90% of those <65 years (Fukuda et al., 1999). The currently available trivalent inactivated influenza vaccines are particularly ineffective in preventing deaths among elderly persons with associated chronic conditions (Nichol et al., 1998; Gross, 1995), underscoring the need for influenza vaccines that are more effective in elderly persons that need them most.

Antibodies specific for influenza surface antigens such as hemagglutinin (HA) and neuraminidase (NA) play an important role in protective immunity when the HA and NA of the vaccines closely resemble those of the circulating virus strains. Mutation of HA and NA can result in viral escape from neutralizing antibodies (antigenic drift). Occasionally, new viruses emerge with novel HA and NA, against which preexisting antibodies are absent in the population (antigenic shift). In these cases, the major histocompatibility complex (MHC) class I-restricted CD8⁺ CTL activity directed to more conserved proteins, such as nucleoprotein (NP), matrix protein, and polymerase proteins may contribute to protective immunity against these potentially pandemic viruses (Gotch et al., 1987; Lukacher et al., 1984; Ulmer et al., 1993). In addition, CD8⁺ CTL activity plays a major role in promoting recovery from severe influenza infection (Oldstone, 1994; Bender et al., 1992; McMicael et al., 1983). Thus, it will be essential for an effective influenza vaccine to be capable of inducing both high titers of neutralizing antibodies and robust CTL activity to influenza.

Earlier studies demonstrated that influenza virus-specific CTL activity was significantly diminished among elderly persons when compared with the young (Mbawuike et al., 1993). Results from other groups also indicated an aged-related impairment in CTL activity, showing that elderly persons exhibited significantly lower and shorter-lived CTL response after vaccination with inactivated influenza vaccines (Powers and Belshe, 1993; Powers, 1993). Therefore, diminished CTL activity in the elderly may be responsible for poor protection against influenza infection, leading to occurrence of prolonged and more sever infection in the elderly. This age-related deficiency in CTL activity has also been revealed in animal studies by showing delayed development and reduced activity of CTL responses in aged mice compared to young control mice (Mbawuike et al., 1996; Zhang et al., 2000; Po et al., 2002). Thus improvement of the virus-specific CTL responses in the aged may lead to reduced severity of viral infection in this age group.

Fc receptors (FcRs) link the humoral and cellular branches of the immune system and have important functions in the activation and modulation of immune responses. Since both effector cells such as B cell and mast cells, as well as antigen-presenting cells (APCs), such as dendritic cells (DCs), follicular dendritic cells (FDCs) and B cells, express various types of FcRs, immune complex (IC) can exert their immunoregulatory functions by direct signaling effector cells and/or by targeting APCs. Thus, advantages of ICs in inducing immune responses are several fold: ICs can direct activately effector cells; ICs are effectively taken up by professional APCs; and IC-binding to FcR can act as a natural adjuvant and mediate DC maturation.

The present invention provides a long-felt need in the art by disclosing methods and reagents suited for inducing immune responses, particularly in individuals having compromised immune systems.

### BRIEF SUMMARY OF THE INVENTION

The present invention is directed to a system, methods, and compositions that improve immunity in any individual, including an immune-compromised individual.

In certain aspects of the invention, there are methods and compositions that concern improving immune response in an individual, for example an immune-compromised individual. In particular cases, the invention concerns correction of age-associated deficiency in antibody responses including germinal center reaction by immunization with immune complexes. In other aspects of the invention, there are provided methods and compositions for rectification of age-associated deficiency in cytotoxic T Cell response to Influenza A Virus by immunization with immune complexes.

The present invention also generally concerns the improved function of an aged immune system by Fc receptor signaling and IC immunization overcoming at least age-related immune deficiency including diminished CTL responses. This has been characterized using the exemplary embodiments related to efficacy of IC vaccination in inducing CTL activity against influenza virus. In the invention, immunization with the exemplary ICs significantly enhances immune responses in aged mice. In particular, the data demonstrate that IC consisting of influenza vaccine and monoclonal antibody (mAb) specific for influenza A nucleoprotein can largely overcome the impairment in immune response to influenza and elicit significantly improved CTL responses in aged mice.

In one embodiment, there is a method of improving an immune response in an immune-compromised individual, comprising delivering to the individual a therapeutically effective amount of an immune complex comprising: 1) an antigen; and 2) an antibody that immunologically recognizes the antigen. In a specific embodiment, the antigen is selected from the group consisting of a viral antigen, a bacterial antigen, or a fungal antigen. In another specific embodiment, the viral antigen comprises an inactivated or attenuated intact viral particle. In a further specific embodiment, the viral antigen comprises part or all of a protein of the virus, and in additional specific embodiments, the viral antigen is from Influenza, HIV, Hepatitis, SARS, or Varicella zoster virus.

In a specific embodiment, the bacterial antigen comprises killed or attenuated whole bacteria. In another specific embodiment, the bacterial antigen comprises all or part of a protein of a bacteria. In further specific embodiments, the bacterial antigen is from Staphylococcus, Haemophilus, Streptococcus, Escherichia, Salmonella, Shigella, Yersinia, Klebsiella, Pseudomonas, Enterobacter, Salmonella, Serratia, or Proteus.

In another specific embodiment, the antigen comprises part or all of a fungal protein. In an additional specific embodiment, the antigen is from Candida, Aspergillus, Cryptococcus, Coccidioides, Histoplasma, Pneumocystis, or Paracoccidioide.

An individual suitable for receiving the invention is elderly, very young (for example, infants, toddlers, or children), has an infection, is being treated for cancer, has genetic immune deficiencies, inherent immune deficiencies, and/or has had an organ or tissue transplant, for example.

In one embodiment, there is a kit for an immune-compromised individual, comprising an immune complex, said kit housed in a suitable container and comprising 1) an antigen; and 2) an antibody that immunologically recognizes the antigen. In a specific case, the antigen is selected from the group consisting of a bacterial antigen, fungal antigen, or viral antigen.

In an additional embodiment, there is an isolated antibody/antigen immune complex.

In specific embodiments of the present invention, the anti-(4-hydroxy-3-nitrophenyl)acetyl (NP) response in aged mice is characterized after immunization with ICs consisting of NP-specific monoclonal antibody and NP-chicken γ-globulin (CGG) conjugate. In mice of the IgH^{b} allotype, the antibody response to NP is highly restricted. Most primary NP-specific antibodies bear the λ₁ L chain and are encoded by the V_{H}186.2 gene segment (Bothwell et al., 1981). The results demonstrate that diminished GC response in aged animals can be significantly restored by IC immunization. In addition, memory antibody response and long-lived plasma cells in the bone marrow were significantly enhanced in aged mice immunized with ICs, indicating a recovery of a functional GC reaction. The findings indicate that an impaired GC response in aging can be largely overcome by IC immunizations, which provides useful vaccines and immunization protocols for immune-compromised individuals.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows that immunization with immune complex restores the age-related defect in GC formation. Young (3 month) or aged (24 month) C57BL/6 mice were immunized with antigen (NP-CGG), NP-CGG plus isotype control antibody, or immune complex consisting of NP-CGG and NP-specific antibody. The primary (FIG. 1A) and secondary (FIG. 1B) GC volume formed in young (black bars) and aged (open bars) mice was determined planometrically on photographs of splenic sections of immunohistologic staining. Data (mean±SE) are representative of 3 independent experiments with 5 mice in each group.

FIG. 2 demonstrates that immune complex immunization enhances class-switched NP-specific antibody response in a primary immune response. Sera from aged (FIG. 2A) or young (FIG. 2B) mice immunized with NP-CGG alone, NP-CGG/control antibody, or NP-specific ICs were measured by ELISA for NP-specific IgG1 antibodies. Data (mean±SE) are representative of 3 independent experiments with 5 mice in each group.

FIG. 3 shows that immune complex immunization enhances secondary antibody response. Sera from aged (FIG. 3A) or young (FIG. 3B) mice immunized with NP-CGG alone (open squares), NP-CGG/control antibody (closed circles), or NP-specific ICs (closed squares) collected in the secondary response at time points indicated were measured by ELISA for NP-specific IgG1 antibodies. Data (mean±SE) are representative of 3 independent experiments with 5 mice in each group. Asterisk indicates p<0.05 between experimental and control groups.

FIG. 4 shows that immune complex immunization increases the number of long-lived BM AFCs. Twelve days post secondary immunization, BM AFCs from aged (FIG. 4A) or young (FIG. 4B) mice were enumerated from ELISPOT. High-affinity (NP5-binding, open bars) or total (NP25-binding, black bars) AFCs were detected. Data (mean±SE) are representative of 3 independent experiments with 5 mice in each group.

FIG. 5 demonstrates that immune complex immunization enhances T-cell priming in vivo. C57BL/6 mice were immunized with NP-CGG/IC (filled symbols) or NP-CGG/control Ab (open symbols) s.c. Seven days later, cellular proliferation of draining LN cells against recall NP-CGG was measured. Cells were harvested 96 hours later in the presence of H3-thymidine for the last 18 hours. Data (mean±SE) are representative of 2 independent experiments with 3 mice in each group. Asterisk indicates p<0.05 between experimental and control groups. FIG. 5A is data from the aged group and FIG. 5B is data from the young group.

FIG. 6 shows that immune complex vaccination enhances CTL responses against influenza virus-infected target cells in aged mice. Young (FIG. 6A) or aged (FIG. 6B) BALB/c mice were immunized with live influenza virus (black circles), inactivated virus, i.p. (open squares), inactivated virus with isotype control antibody (black squares), or immune complex vaccine (black diamonds), as described in the text. 5 weeks later, mice received the same injections for each group. Twelve days after boost, spleen cells were prepared and stimulated for 6 days with virus-infected syngeneic spleen cells (upper panels in both figures), or medium only (lower panels). Cells were then washed and titrated in the specific cytotoxicity assay. Target cells were P815 (H-2^{d}) cells infected with live virus (left columns in both figures), P815 cells exposed to medium only (middle columns), or EL-4 (H-2^{b}) cells infected with virus (right columns). Cytotoxicity was determined after 4 hrs. by measuring released 51 Cr. Data (mean±s.e.) are from triplicate assays from an experiment with 6 mice in each group. Similar independent experiments have been repeated twice.

FIG. 7 demonstrates that immune complex vaccination enhances IFN-γ production by CD8⁺ T cells responding to influenza A immunization in aged mice. Immunization of aged mice and stimulation of splenic cells are described in FIG. 6. 4 days after in vitro stimulation with influenza virus-infected splenic cells, cultured cells from mice immunized with live virus, vaccine only, immune complex vaccine, or vaccine plus isotype control antibody were harvested and stained for intracellular LFN-γ. (FIG. 7A) Profiles of CD8 and IFN-γ staining in lymphocyte gate. Numbers in individual samples indicate % of CD8⁺IFN-γ⁺ cells in total lymphocyte gate. (FIG. 7B) Percentage (mean±s.e.) of IFN-γ⁺ cells in CD8⁺ cell gate. (FIG. 7C) MFI (mean±s.e.) of IFN-γ expression in individual samples. Results are representative of two independent experiments.

FIG. 8 shows that immune complex vaccination enhances antibody and AFC responses to influenza A in aged mice. Aged BALB/c mice were immunized with live virus, vaccine, vaccine plus isotype control antibody, or vaccine IC. Sixteen days after immunization, serum antibody levels and numbers of splenic or bone marrow AFCs were analyzed. (FIG. 8A) serum IgG antibodies specific for HA (open bars) and NP (black bars) were determined by ELISA. An anti-NP monoclonal antibody and a pool of HA positive sera were used as standard for NP- or HA specific antibodies, respectively. (FIG. 8B) Bone marrow (solid bars) or splenic (open bars) IgG₁ AFCs secreting specific antibodies against influenza A virus were determined by ELISPOT assay. Data (mean±s.e.) are from an experiment with 6 mice in each group. Similar independent experiments have been repeated twice.

FIG. 9 demonstrates that immune complex immunization promotes Th1 cytokine production. Seven days after immunization, draining lymph node cells from mice primed with immune complex (black bars) or vaccine plus isotype control antibody (open bars) were stimulated with influenza A vaccine for three days. Cytokine profiles were determined by intracellular cytokine staining. Asterisks indicate p<0.05. Data (mean±s.e.) were percentages of cells positive for individual cytokines in total lymphocyte gate. Similar results were obtained from three independent experiments. FIG. 9A is % INF, FIG. 9B is IL-4, and FIG. 9C is IL-10.

FIG. 10 demonstrates that immune complex enhances dendritic cell maturation and function. Purified dendritic cells were cultured with immune complex vaccine or vaccine mixed with isotype control antibody for 48 hours. (FIG. 10A) Percentages of CD 11c⁺ dendritic cells with different levels of MHC II or CD86 expression are shown. (FIG. 10B) Mean fluorescence intensity (MFI) of MHC II or CD86 expression on dendritic cells under different culture conditions are shown. Data are representative of three independent experiments.

FIG. 11 demonstrates that immune complex immunization enhances GC response to HIV-1. GC responses were analyzed at day 12 after immunization with IC or control immunogen. FIG. 1A shows the draining lymph node (LN) cells were stained with anti-B220-APC, GL-7-FITC and anti-Fas-PE and analyzed by FACS. Numbers represent the percentages of GL7⁺Fas⁺ GC B cells within the B cell (B220⁺) gate). FIG. 11B shows the percentages of splenic GC B cells analyzed by FACS. FIG. 11C shows fluorescence microscopic pictures showing GCs formed in the follicles in the spleen from control or IC-immunized mice. Sections were stained with PNA-FITC (green, revealing GCs) and anti-IgD-PE (red, visualizing B cell follicles). N=5. Similar experiments were repeated twice.

FIG. 12 shows that immune complex immunization promotes the production of anti-gp120 antibodies. FIG. 12A measures IgM, and FIG. 12B measureds IgG. Serum samples were collected at day 12 post-immunization and analyzed by ELISA. The plates were coated with 5 μg gp120 protein (NIH AIDS Research & Reference Reagent Program). Sera were diluted at 1:500, *p<0.05, and n=5. Data (mean±s.e.) were representative of two independent experiments with similar results.

FIG. 13 shows that immune complex immunization enhances anti-gp120 AFC responses. Splenic and lymph node (LN) cells were collected at day 12 post-immunization and analyzed by ELISPOT assay. Membrane filters were coated with gp120 (10 μg/ml) in PBS overnight at 4° C. Data (mean±s.e.) were representative of two independent experiments with similar results. n=5. Student t test was used. P values are shown in each figure.

FIG. 14 demonstrates immunization with ICs enhances T-cell priming to HIV-1. Twelve days after immunization, draining LN cells from IC-immunized mice (solid circles) or controls (open circles) were stimulated with various concentrations of virions for 3 days. Cellular proliferation was measured by H³-thymidine incorporation. Five mice were in each group. Data (mean±s.e.) are representative of two independent experiments.

FIG. 15 shows that immunization with ICs enhances Ab memory response. Samples were collected from IC-immunized (closed circles) mice or controls (open circles) at various days after secondary immunization as indicated. Five mice were in each group. Data (mean±s.e.) are representative of two independent experiments.

FIG. 16 demonstrates that immunization with ICs enhances B-cell priming. Twelve days after secondary immunization, purified B cells from IC-immunized mice (closed circles) or controls (open circles) were stimulated with various concentrations of virions for 4 days. Cellular proliferation was measured by H³-thymidine incorporation. Data (mean±s.e.) are from an experiments with 5 mice in each group.

FIG. 17 shows that IC immunization significantly increased serum neutralization titers. Sera from mice immunized with IC containing HIV-1 (97ZA012) and anti-gp120 mAb or controls were diluted and subjected to neutralization assay using PBMC infected with ZA012. FIG. 17A shows the results were shown as the serum neutralization IC₅₀, which is the reciprocal of the serum dilution producing 50% neutralization. The IC samples showed ˜10-fold higher neutralization activity compared to controls. FIG. 17B shows the percent of neutralization vs. serum dilution was plotted to show 2 control samples (Ctrl-1 and Ctrl-2) and two IC samples (IC-1 and IC-2). IC samples show nice dose-dependent curves.

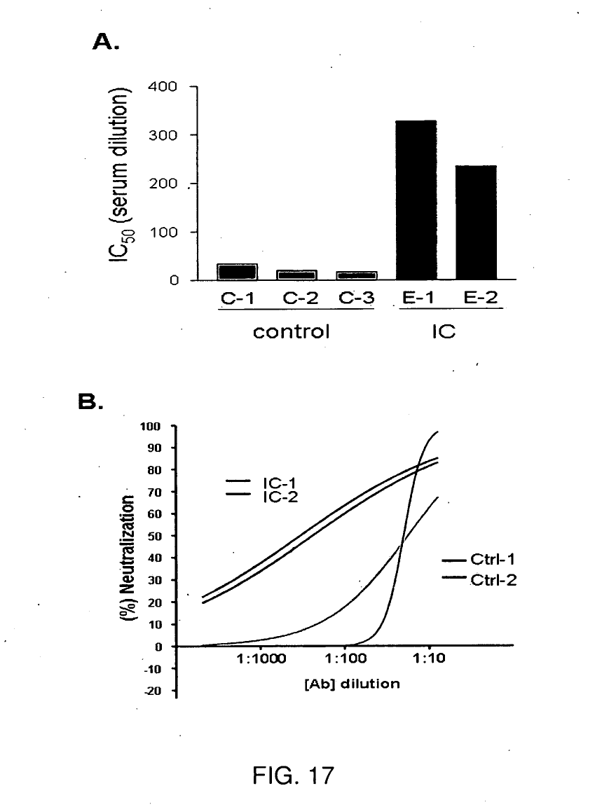

FIG. 18 demonstrates IC immunization enhances anti-gp120 AFC responses in CD4−/− mice. Splenic cells were isolated at day 12 post-immunization. IgM- or IgG-AFCs were determined by ELISPOT. The frequencies of both isotypes of AFCs were significantly increased after IC immunization. The data are representative of two independent experiments. FIG. 18A measures splenic IgM-AFCs, while FIG. 18B. measures splenic IgG-AFCs.

### DETAILED DESCRIPTION OF THE INVENTION

### I. Exemplary Definitions

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “immune complex” as used herein refers to antigen:antibody complexes, which are formed through the interactions between antigens and antibodies.

The term “immune-compromised” or “immune-deficient” as used herein refers to individuals have diseases or conditions that are failures of host defense against infection, in which one or more components of the immune system (B cells, T cells, phagocytes, complement molecules, etc.) is functionally defective (due to genetic defects, development, aging, infection, physical/chemical agents including radiation/chemotherapy, etc.), leading to heightened susceptibility to infection.

The term “immunologically recognizes” as used herein refers to recognition conferred by molecules of the immune system, such as T-cell receptor (TCR), B-cell receptor (BCR), major histocompatibility complex (MHC) molecules, or toll-like receptors (TLR), for example.

The term “improvement,” “improving,” or any variants as used herein refer to improving immune response. The improvement is any observable or measurable improvement. The improvement may be in just one aspect of immune response, or may be a combination of factors. Non limiting factors associated with improving immune response may be increased GC volume, increased levels of antibody production, or increased number and life-span of plasma cells or memory cells, for example. One of skill in the art knows methods and materials needed to measure or observe improvement in immune response.

The term “vaccine” as used herein refers to dead or actuated forms of pathogen or components of a pathogen that is administered to an individual to deliberately induce adaptive immunity to the pathogen, while the term “immunological composition” refers to any composition that induces one or more immune responses.

### II. Embodiments of the Invention

The present invention, in particular embodiments, concerns immunization with immune complexes for any individual, including individuals that are immune-compromised. In particular embodiments, the individuals are immune-compromised due to age, although in other embodiments the individuals are immune-compromised due to infection or illness.

In aging, for example, both primary and secondary antibody responses are impaired. One of the most notable changes in age-associated immune deficiency is the diminished germinal center (GC) reaction. This impaired GC response reduces antibody affinity maturation, decreases memory B cell development, and prevents the establishment of long-term antibody-forming cells in the bone marrow. The present invention generally concerns immunization with immune complexes in overcoming deficiencies in GC response, including age-associated deficiency in GC response. It is shown herein that the depressed GC response in aged mice, as a model for mammals, can be significantly elevated by immunization with immune complexes. Importantly, there is a significant improvement of B cell memory response and long-lived plasma cells. The results demonstrate that immune complex immunization is useful to elicit functional GC response in aging and to overcome age-related immune deficiency in general.

In other embodiments of the invention, decline in cellular immunity in aging compromises protection against infectious diseases and leads to the increased susceptibility of the elderly to infection. For example, antigen-specific cytotoxic T lymphocyte (CTL) response against virus is markedly reduced in an aged immune system. The present invention concerns the efficacy and mechanisms of immunization with immune complexes in overcoming age-associated deficiency in cellular immunity. It is shown herein that the severely depressed CTL response to influenza A in aged mice can be significantly restored by immunization with immune complexes comprising of influenza A virus and monoclonal antibody to influenza A nucleoprotein. The main mechanisms underlying this recovery of CTL response induced by immune complex immunization in aged mice are enhanced dendritic cell function and elevated production of IFN-γ in both CD4⁺ Th1 and CD8⁺ cytotoxic T cells. Thus, these results demonstrate that immune complex immunization is useful to elicit effective virus-specific cytotoxic response in an aged immune system and to overcome age-related immune deficiency in general.

### III. Embodiments of Immune Complexes

In particular aspects of the invention, immune complexes are delivered to an individual that is immune-compromised. In specific aspects, the immune complexes are isolated from nature, whereas in further specific aspects the immune complexes are generated in vitro (for vaccination, the immune complexes may be pre-formed in vitro).

Exemplary antigens and antibodies of the immune complexes are provided below. One of skill in the art would be able to determined other immune complexes of the present invention.

A. Antigens

The antigens of the present invention may be of any kind, but in particular cases they are viral antigens, bacterial antigens, or fungal antigens. An individual may be administered with more than one kind of immune complex, including separate immune complexes to one kind of antigen (for example, a viral antigen) and to another kind of antigen (for example, a bacterial antigen).

1. Viral Antigens

The viral antigens of the immune complexes of the invention may be of any kind, but in particular cases they include the following, for example: (1) Influenza, including inactivated or attenuated intact viral particles; for Influenza A: nucleoprotein (NP), hemagglutinin (HA), neuraminidase (NA), or M2 protein; for Avian influenza A (H₅N₁): H5; (2) HIV, including inactivated intact viral particles; structural genes and proteins (gag, env, gp120, gp41 and gp160); viral enzyme (pol); or regulating proteins (nef, tat, rev and vpr); (3) Hepatitis viruses, including HBV (Inactivated viral particles, recombinant vaccine); HCV (inactivated viral particles, recombinant vaccine); or (4) VZV (varicella-zoster virus), including inactivated or attenuated intact viral particles.

2. Bacterial Antigens

The bacterial antigens of the immune complexes of the invention may be of any kind, but in particular embodiments they comprise one of the following, for example: (1) Staphylococcus aureus, including killed whole bacteria; capsular polysaccharides (such as type 5 and type 8) coupled to carriers (such as pseudomonas exotoxin A toxoid); or protein A; (2) Haemophilus influenzae, including killed whole bacteria; capsular polysaccharide of Hib conjugated to carrier such as tetanus toxoid; (3) Streptococcus pneumoniae, including killed whole bacteria; polysaccharide conjugated to carrier such as tetanus toxoid; or (4) Enteric gram-negative pathogens, including killed or attenuated whole bacteria (Escherichia, Salmonella, Shigella, Yersinia, Klebsiella, Pseudomonas, Enterobacter, Salmonella, Serratia, Proteus); enterotoxins.

3. Fungal Antigens

The fungal antigens of the immune complexes of the invention may be of any kind, but in particular embodiments they comprise one of the following, for example: (1) Candida spp., including algal β-glucan (laminarin) conjugated with a protein component such as tetanus toxoid; HSP90; (2) Aspergillus spp., including algal β-glucan (laminarin) conjugated with a protein component such as tetanus toxoid; Aspf16; (3) Cryptococcus neoformans, including Glucuronxylomannan (GXM) and GXM peptide mimotopes; polysaccharide deacetylase; (4) Coccidioides spp., including proline-rich antigen; (5) Histoplasma capsulatum, including Histone-H₂B-like protein; Hsp60; (6) Pneumocystis carinii, including Major surface glycoprotein; p55; (7) Paracoccidioides brasiliensis, including gp43; BAD 1.

B. Antibodies

The immune complexes of the invention may be of any kind, but in particular aspects they employ monoclonal or polyclonal antibodies or fragments thereof. In specific embodiments, there are antibody fragments such as Fab or Fab2, but in alternative embodiments the antibodies comprise the Fc portion of the antibody. In further specific embodiments, they should be purified by various methods as needed, such as, for example, affinity chromatography, size-exclusion chromatography, or ion-exchange chromatography.

### IV. Production and Delivery of the Immune Complexes

Immune complexes may be produced by mixing antigens and antibodies at certain ratios according to the size of the antigen and the number of antibody-binding sites on each antigen. The immune complexes may be given intramuscularly, intradermally, or subcutaneously, for example.

### V. Immune-Compromised Individuals

In certain aspects of the invention, the immune complexes are provided to an immune-compromised individual, although in additional or alternative embodiments the immune complexes are provided to an individual that is not immune-compromised.

In immune-compromised individuals, the ability of the body's immune system to respond is decreased. This condition may be present at birth, or it may be caused by certain infections (such as human immunodeficiency virus or HIV, for example), or by certain cancer therapies, such as cancer-cell killing (cytotoxic) drugs, radiation, and bone marrow transplantation, for example. In fact, prevention or interference with the development of an immunologic response may occur by any manner, and may reflect natural immunologic unresponsiveness (tolerance); may be artificially induced by chemical, biological, or physical agents, or may be caused by disease.

Examples of immune-compromised individuals include the elderly, which may be defined as an individual that is about 65 years of age or older, an individual infected with HIV, an individual with AIDS, an individual taking chemotherapy or immunosuppressants, and so forth.

In specific embodiments, immunosuppression may be considered a symptom in the following exemplary conditions, and therefore individuals with one or more of at least these conditions are suitable for delivery of the immunocomplexes of the invention: aspergillosis, HIV, hematopoietic stem cell transplant, granulocytopenia, organ transplant recipients, avascular necrosis, Bacterial meningitis, Candidiasis, AIDS, immunosuppressive medications, cervical cancer, Coccidioidomycosis, Cryptococcosis, Cryptosporiosis, depression, Diarrheagenic Escherichia coli, Ehrlichiosis, Endocarditis, Flu, Food poisoning, Fungal infections, Fungal meningitis, Group A Streptococcal Infections, Group B Streptococcal Infections, Invasive candidiasis, Invasive group A Streptococcal disease, Kaposi's Sarcoma, Legionnaires' disease, Leukemia, Listeriosis, Melanoma, Melioidosis, Meningitis, Meningococcal disease, Molluscum contagiosum, Mycobacterial infections, Mycobacterium avium Complex, Nocardiosis, Non-Hodgkin's Lymphoma, Nosocomial infections, Oral thrush, Pneumococcal meningitis, Pneumococcal pneumonia, Pneumococcus, Pneumonia, Pontiac fever, Pyelonephritis, Q fever, Respiratory syncytial virus, Salmonella enteritidis, Salmonella food poisoning, Septicemia, Shingles, Staphylococcal infection, Streptococcal Infections, Toxoplasmosis, Traveler's diarrhea, Tuberculosis, Vaginal candidiasis, Vibrio parahaemolyticus, and Vibrio vulnificus.

Thus, in immune-compromised individuals (aged people, HIV individuals, cancer individuals receiving radiation/chemotherapy, and transplant recipients receiving immune suppression drugs, for example) are susceptible for these infectious diseases and are candidates for receiving IC immunological composition against the above pathogens. In another embodiment of the invention, the individual is not immuno-compromised.

### VI. Immunological Reagents

In certain aspects of the invention, one or more antibodies may be produced that recognize the antigen of the immune complex. As used herein, the term “antibody” is intended to refer broadly to any immunologic binding agent such as IgG, IgM, IgA, IgD and IgE. In one embodiment, IgG and/or IgM are used because they are the most common antibodies in the physiological situation and because they are most easily made in a laboratory setting and are likely to be effective in modulating the immune system for vaccination.

The term “antibody” is used to refer to any antibody-like molecule that has an antigen binding region, and includes antibody fragments such as Fab′, Fab, F(ab′)2, single domain antibodies (DABs), Fv, scFv (single chain Fv), and the like and are likely to be effective in modulating the immune system for vaccination. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and may be used. The invention thus provides monoclonal antibodies of the human, murine, monkey, rat, hamster, rabbit and even chicken origin. Due to the ease of preparation and ready availability of reagents, murine monoclonal antibodies may also be used, in certain embodiments.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's disease are likewise known and such custom-tailored antibodies are also contemplated.

The methods for generating monoclonal antibodies (MAbs) generally begin along the same lines as those for preparing polyclonal antibodies. Briefly, a polyclonal antibody is prepared by immunizing an animal with an antigen composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, an interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as beta-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or by cardiac puncture, for example. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats maybe used, as well as rabbit, sheep or frog cells. The use of rats may provide certain advantages (Goding, 1986, pp. 60 61), but mice are also used, with the BALB/c mouse being routinely used and generally give a higher percentage of stable fusions.

The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells may be used, the former because they are a rich source of antibody-producing cells that are in the dividing plasmablast stage, and the latter because peripheral blood is easily accessible, however, the use of other somatic cells is also considered.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody producing B lymphocytes from the immunized animal are then fused with cells of an immortal myeloma cell, generally one of the same species as the animal that was immunized. In one embodiment, myeloma cell lines suited for use in hybridoma producing fusion procedures are non antibody producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65 66, 1986; Campbell, pp. 75 83, 1984). For example, where the immunized animal is a mouse, one may use P3×63/Ag8, X63 Ag8.653, NS1/1.Ag 4 1, Sp210 Ag14, FO, NSO/U, MPC 11, MPC11 X45 GTG 1.7 and S194/5XXO Bul; for rats, one may use R210.RCY3, Y3 Ag 1.2.3, IR983F and 4B210; and U 266, GM1500 GRG2, LICR LON HMy2 and UC729 6 are all useful in connection with cell fusions.

One such murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8 azaguanine resistant mouse murine myeloma SP2/0 non producer cell line.

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71 74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

One selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. In one embodiment, the assay can be be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

### VII. Antibody Conjugates

The present invention further provides antibodies to antigens, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti cellular agent, and may be termed “immunotoxins”.

Antibody conjugates are may be used as diagnostic agents. Antibody diagnostics generally fall within two classes, those for use in in vitro diagnostics, such as in a variety of immunoassays, and/or those for use in vivo diagnostic protocols, generally known as “antibody directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) erbium (III), and/or gadolinium. Ions useful in other contexts, such as X-ray imaging, include but are not limited to lanthanum (III), gold (III), lead (II), and bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being used in certain embodiments, and technicium^{99m} and/or indium¹¹¹ are also often used due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Other secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulflhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### VIII. Vaccines of the Invention

For an antigenic composition to be useful as a vaccine, an antigenic composition must induce an immune response to the antigen in an animal (e.g., a human). As used herein, an “antigenic composition” comprises an immune complex. In some embodiments, the immune complex is in a mixture that comprises an additional immunostimulatory agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

In certain embodiments, an antigenic composition or immunologically functional equivalent, may be used as an effective vaccine in inducing an anti-immune complex humoral and/or cell mediated immune response in an animal. The present invention contemplates one or more antigenic compositions or vaccines for use in both active and passive immunization embodiments.

A vaccine of the present invention may vary in its composition of nucleic acid, proteinaceous, cellular, and/or whole pathogen components. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

### IX. Proteinaceous Antigens

It is understood that an antigenic composition of the present invention may be made by a method that is well known in the art, including but not limited to chemical synthesis by solid phase synthesis and purification away from the other products of the chemical reactions by HPLC, for example, or production by the expression of a nucleic acid sequence (e.g., a DNA sequence) encoding a peptide or polypeptide comprising an antigen of the present invention in an in vitro translation system or in a living cell. The antigenic composition may be isolated and extensively dialyzed to remove one or more undesired small molecular weight molecules and/or lyophilized for more ready formulation into a desired vehicle. It is further understood that additional amino acids, mutations, chemical modification and such like, if any, that are made in a vaccine component may not substantially interfere with the antibody recognition of the epitopic sequence.

A peptide or polypeptide corresponding to one or more antigenic determinants of the antigen part of the immune complex of the present invention should generally be at least five or six amino acid residues in length, and may contain about 10, about 15, about 20, about 25, about 30 about 35, about 40, about 45 or about 50 residues or more. A peptide sequence may be synthesized by methods known to those of ordinary skill in the art, such as, for example, peptide synthesis using automated peptide synthesis machines, such as those available from Applied Biosystems (Foster City, Calif.).

Longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present invention. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell.

### X. Genetic Vaccine Antigens

In certain embodiments, an immune response may be promoted by transfecting or inoculating an animal with a nucleic acid encoding an antigen. One or more cells comprised within a target animal then expresses the sequences encoded by the nucleic acid after administration of the nucleic acid to the animal. Thus, the vaccine may comprise “genetic vaccine” useful for immunization protocols. A vaccine may also be in the form, for example, of a nucleic acid (e.g., a cDNA or an RNA) encoding all or part of the peptide or polypeptide sequence of an antigen. Expression in vivo by the nucleic acid may be, for example, by a plasmid type vector, a viral vector, or a viral/plasmid construct vector.

In one embodiment, the nucleic acid comprises a coding region that encodes all or part of an antigen, or an immunologically functional equivalent thereof. Of course, the nucleic acid may comprise and/or encode additional sequences, including but not limited to those comprising one or more immunomodulators or adjuvants. The nucleotide and protein, polypeptide and peptide encoding sequences for various genes have been previously disclosed, and may be found at computerized databases known to those of ordinary skill in the art. One such database is the National Center for Biotechnology Information's Genbank and GenPept databases. The coding regions for these known genes may be amplified and/or expressed using the techniques disclosed herein or by any technique that would be know to those of ordinary skill in the art (e.g., Sambrook et al., 1987). A nucleic acid may be expressed in an in vitro expression system, in other embodiments the nucleic acid comprises a vector for in vivo replication and/or expression.

### XI. Cellular Vaccine Antigens

In another embodiment, the immune complex of the vaccine may comprise a cell, such as a bacterial or fungal organism, or an intact viral particle. The cell may be isolated from a culture, for example, and administered to an animal as a cellular vaccine. Thus, the present invention contemplates a “cellular vaccine.” The cell may be transfected with a nucleic acid encoding an antigen to enhance its expression of the antigen. Of course, the cell may also express one or more additional vaccine components, such as immunomodulators or adjuvants. A vaccine may comprise all or part of the cell.

### XII. Pharmaceutical Preparations

Pharmaceutical compositions of the present invention comprise an effective amount of one or more immune complexes dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one immune complex will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The immune complex may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The immune complex may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include immune complex, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the immune complexes may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In certain embodiments of the present invention, the immune complexes are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet, for example.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and about 1% to about 2%.

B. Parenteral Compositions and Formulations

In further embodiments, immune complexes may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents may be included, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, in one embodiment the methods of preparation are vacuum-drying or freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other embodiments of the invention, the active compound immune complexes may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### XIII. Exemplary Kits of the Invention

Any of the compositions described herein may be comprised in a kit. In a non-limiting example, an immune complex, and in some cases an additional agent, may be comprised in a kit. The kits will thus comprise, in suitable container means, one or more immune complexes of the invention. The kits may comprise a suitably aliquoted immune complexes, and the components of the kits may be packaged either in aqueous media or in lyophilized form, for example. The container means of the kits will generally include at least one vial, test tube, flask, bottle, syringe or other container means, into which a component may be placed, and suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the immune complexes, additional agents, and any other reagent containers in close confinement for commercial sale. Such containers may include injection or blow-molded plastic containers into which the desired vials are retained.

The components of the kit may be provided as dried powder(s). When reagents and/or components are provided as a dry powder, the powder can be reconstituted by the addition of a suitable solvent. It is envisioned that the solvent may also be provided in another container means. The kits may comprise a second container means for containing a sterile, pharmaceutically acceptable buffer and/or other diluent.

Irrespective of the number and/or type of containers, the kits of the invention may also comprise, and/or be packaged with, an instrument for assisting with the injection/administration and/or placement of the ultimate immune complex composition within the body of an animal. Such an instrument may be a syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Exemplary Materials and Methods for Examples 2-5

The present example concerns exemplary materials and methods related to some embodiments of the invention.

### Mice

Young (2-4 months old) and aged (20-24 months old) C57BL/6J (H-2b) mice were from Charles River (Wilmington, Mass.) from cohorts maintained by the National Institute on Aging, NIH. All animals were maintained in autoclaved microisolator cages, and provided with sterile bedding, food and water. Animal experimentation was performed in accordance with protocols approved by IACUC of Baylor College of Medicine.

### Antigens, Immune Complex Formation, and Immunization

Hapten NP (Cambridge Research Biochemicals, Cambridge, UK) was conjugated to CGG (Accurate Chemical & Scientific, Westbury, N.Y.) as described (Weinberger et al., 1979). The final conjugation ratio was NP₂₂/CGG. The NP-specific monoclonal antibody (IgG1) was purified by a protein G kit (Pierce, Rockford, Ill.) from supernatants of a transfectoma pEV_{H}Cγ1/γ1 (Allen et al., 1988), which is encoded by germ-line, canonically rearranged V186.2-DFL16.1-JH2 heavy-chain genes and germ-line λ1 light-chain gene. This IgG₁ mAb binds to NP with the dissociation constant K_{d}=1·2×10 6 m. IgG₁ isotype control monoclonal antibody was purchased from Serotec (CRL-1818). Immune complexes or control mixtures were prepared by incubating equal amount of antigen and antibody at 37° C. for 2 hours, then at 4° C. for 18 hours. For primary immunization, mice were immunized i.p. with a single does of the following preparations: 100 μg NP-CGG in PBS, immune complex containing 100 g NP-CGG, or 100 μg NP-CGG with isotype control antibody. For secondary immunization, the same injections were given 60 days later.

### Immunohistology

Spleens were fresh frozen in OCT embedding media; serial, 6-μm thick frozen sections were cut in a cryostat microtome, thaw mounted onto poly-L-lysine-coated slides, air-dried, fixed in ice-cold acetone for 10 min and stored at −80° C. (Han et al., 2003; Zheng et al., 1996; Jacob et al., 1991). Immunolabeling of tissue sections was performed as described (Han et al., 2003; Zheng et al., 1996; Jacob et al., 1991). Briefly, splenic GC were labeled by peanut agglutinin (PNA) conjugated to horseradish peroxidase (HRP; E-Y Laboratories, San Mateo, Calif.) or by biotinylated GL-7 antibody followed by streptavidin-HRP (Southern Biotechnology Associates, Birmingham, Ala.). Bound HRP activity was then visualized using 3-aminoethyl carbazol as previously described (7). The splenic GC volume formed after immunization was determined planometrically on photographs of splenic sections as described (Han et al., 1995).

### Measurement of Antibody-Forming Cells (AFC)

The frequencies of NP-specific AFC from both splenocytes and bone marrow (BM) cells were estimated by ELISPOT assay using two different coupling ratios of NP-BSA as described (Han et al., 2003). Briefly, nitrocellulose filters were coated with 50 μg/ml NP5-BSA, NP25-BSA or BSA in PBS at 4° C. overnight and then blocked with 10% FCS in PBS. Splenocytes (5×10⁵ cells/well) or BM cells (1×10⁶ cells/well) were incubated on the filters in 96-well plates at 37° C., 5% CO₂. After 2-hour incubation, filters were washed with PBS containing 50 mM EDTA once, followed by PBS containing 0.1% Tween 20 twice and PBS once. Filters were double-stained with alkaline phosphatase-conjugated anti-mouse IgM and HRP-conjugated anti-mouse IgG1 antibodies. Alkaline phosphatase and HRP activities were visualized using 3-aminoethyl carbazol and napthol AS-MX phosphate/Fast Blue BB respectively. The frequencies of high-affinity and total AFCs were determined from NP5-BSA- and NP₂₅-BSA-coated filters after background on BSA-coated filters was subtracted.

The threshold of antibody affinity which can be detected by each NP-BSA conjugate was determined using several J558L myeloma lines (H⁻, λ₁⁺) transfected with an Igγ₁ expression vector carrying different VDJ rearrangements derived from NP-binding B cells (Dal Porto et al., 1998). Transfectomas secreting NP-binding antibodies with an association constant (Kₐ)=2.0×10⁷ M⁻¹ could be detected by both NP₅-BSA and NP₂₅-BSA. However, transfectomas with Kₐ=10⁶ M⁻¹ could be detected by NP₂₅-BSA, but not by NP₅-BSA. Transfectomas with Kₐ=2.3×10⁵ M⁻¹ could not be detected by either NP-BSA coat. Thus, AFCs secreting antibodies with Kₐ≧2.0×10⁷ M⁻¹ can be detected with NP₅-BSA, while those with Kₐ≧10⁶ M⁻¹ can only be detected with NP₂₅-BSA.

### Measurement of Serum Antibodies

Antibodies specific for the NP hapten were detected by ELISA using two different coupling ratios of NP-BSA as the coating antigens as described (Han et al., 2003). Briefly, 96-well flat-bottom plates (Falcon; Becton Dickinson, Oxnard, Calif.) were coated with 50 μg/ml NP₅-BSA or NP₂₅-BSA in 0.1 M carbonate buffer (pH 9.0) at 4° C. overnight. On each plate, mAb specific for NP, H33Lγ₁/λ₁ (Dal Porto et al., 1998) or B1-8 (Reth et al., 1978) were included as controls. After washing with PBS containing 0.1% Tween 20, HRP-conjugated goat anti-mouse IgG1 or IgM was added and incubated at room temperature for 1 hour. HRP activity was visualized using a TMB peroxidase substrate kit (Bio-Rad, Hercules, Calif.) and optical densities were determined at 450 nm. The concentrations of anti-NP IgG1 or IgM antibodies were calculated by comparison to standard curves created from the H33Lγ₁/λ₁ or B1-8 control antibodies respectively on each plate. To estimate the affinity of NP-binding antibodies in the sera, the ratios of NP₅-binding to NP₂₅-binding antibodies were determined.

### T Cell Priming and In Vitro Re-Call Antigen-Specific T Cell Proliferation

8-week-old female C57BL/6 mice were immunized s.c. at the base of the tail with 100 μg NP-CGG/anti-NP ICs or NP-CGG/control mAb in a volume of 200 μl. Seven days after immunization, draining LN cells were cultured in the presence of various concentrations NP-CGG for 4 days. Cellular proliferation was measured by ³H-thymidine incorporation for the last 18 hours of culture.

### Example 2

### Immunization with Immune Complexes Enhances GC Reaction in Aged Mice

The earlier findings indicate that enhanced B cell response via IC-mediated signals may be the mechanism underlying the recovery of functional GCs in aged mice during a memory response (Han et al., 2004). In this study, it was first investigated whether the diminished GC response in aging can be improved by immunization with pre-formed ICs. Here, GC reaction during both primary and secondary responses was studied following immunization with NP-CGG, ICs, or NP-CGG plus control antibody.

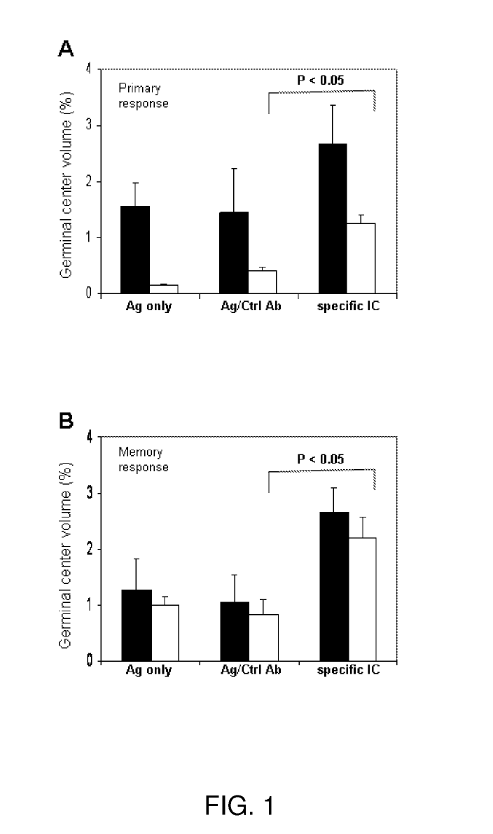

Twelve days after primary or secondary immunization, mice were sacrificed, spleen sections stained with GC markers, PNA and GL-7 antibody. The results showed that the primary GC response in aged mice immunized with the antigen NP-CGG alone or NP-CGG with isotype control antibody was severely diminished compared to that in young mice (FIG. 1A). However, primary GC formation in aged mice was significantly enhanced by IC immunization, to a level comparable to that in young mice immunized with NP-CGG only or NP-CGG plus control antibody (FIG. 1A). Interestingly, the secondary GC formation in aged mice is comparable to that in young mice (FIG. 1B), which is consistent with our earlier findings that secondary immunization significantly enhanced GC response in aged mice (Han et al., 2004). The enhancing effect of ICs on GC formation is also evident in young mice during the memory response. Therefore, the results demonstrate that stimulation with ICs can significantly overcome the age-associated deficiency in GC formation.

### Example 3

### Immunization with IC Corrects Diminished Antibody Responses in Aged Mice

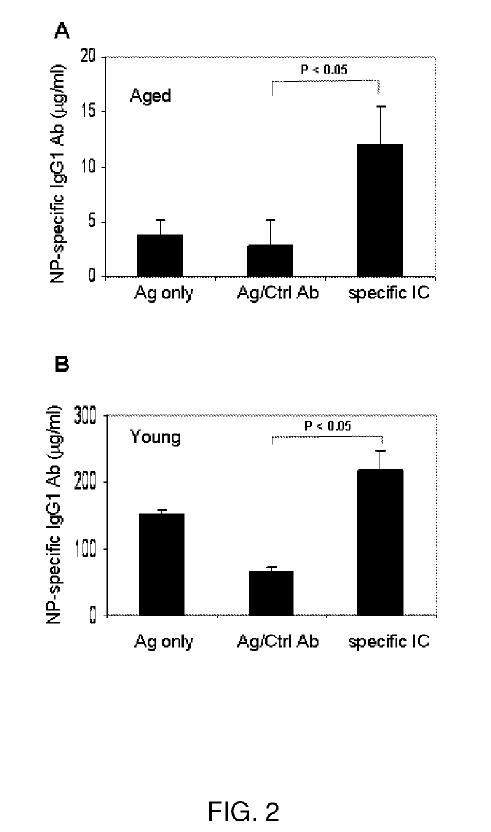

The effects of IC immunization was further evaluated on the age-related defects in antibody response by measuring NP-specific antibodies in primary and secondary immune responses. Following primary immunization, there was a significant increase in IgG₁ NP-specific antibody levels in aged mice immunized with ICs (FIG. 2A). IC immunization also enhanced level of NP-specific IgG₁ antibodies in young mice (FIG. 2B). These finding are consistent with an enhanced GC reaction by IC immunization, since the majority of Ig class-switching takes place during GC response. The level of NP-specific antibody response in aged mice was still very low, about 10% of that in young mice.

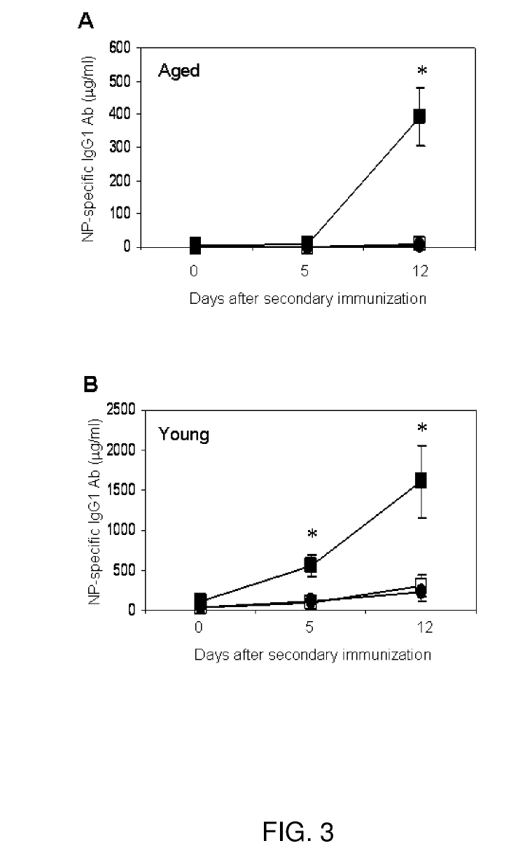

The effects of IC immunization were evaluated on the production of specific antibodies after primary and secondary immunization (Zheng et al., 2007). IC immunization not only increased levels of antigen-specific IgG1 antibodies in aged mice but also in young mice during primary and secondary responses (FIG. 3) (Zheng et al., 2007). At day 12 after challenge, as noted, the levels of NP-specific IgG1 antibodies increased 10-fold and 30-fold in the mice immunized with IC, compared to the mice immunized with antigen alone or antigen with control antibody in young and aged mice respectively (FIG. 3). The IgG1 levels in aged mice immunized with IC (˜300-400 μl/ml) were comparable to that in young mice receiving antigen alone or antigen plus control antibody (FIG. 3). These results demonstrated that not only GC formation was restored in aged mice by IC immunization, but also these IC-induced GCs were functional because GCs are the principal sites for generation of the memory B cells (Liu and Arpin, 1997; Kelsoe 1995), which are responsible for memory antibody responses. IC immunization greatly promotes antibody responses in both young and aged mice.

There was a further increase in NP-specific IgG₁ antibodies in aged mice during the memory response elicited by IC immunization. The amounts of NP-specific IgG₁ antibodies in aged mice in the secondary response induced by IC immunization were significantly increased compared with that induced by antigen alone or antigen with control antibody. By day 12 post secondary immunization, the level of NP-specific IgG₁ antibodies increased more than 30-folds compared to that of primary response (FIG. 3A). Although still lower than that in young mice receiving IC immunization, this level of NP-specific IgG₁ antibodies in aged mice was comparable to that in young mice receiving antigen alone or antigen plus control antibody (FIG. 3B). These results demonstrate that not only GC formation was restored in aged mice by IC immunization, but also these IC-induced GCs were largely functional, since the majority of memory B cell pool is generated during GC response.

### Example 4

### Immune Complex Immunization Promotes Accumulation of Long-Lived Plasma Cells in the Bone Marrow

It has been established that long-lived BM AFCs are derived from GC reaction and responsible for the long-term maintenance of antibody levels (Paramithiotis and Cooper, 1997; Silfka et al., 1995; Manz et al., 1997; Smith et al., 1997; Benner et al., 1981; Benner et al., 1974). To examine antibody-affinity maturation process coupled with the clonal selection in GCs of old mice induced by IC immunization, the levels and affinity of BM AFCs were measured in a memory response.

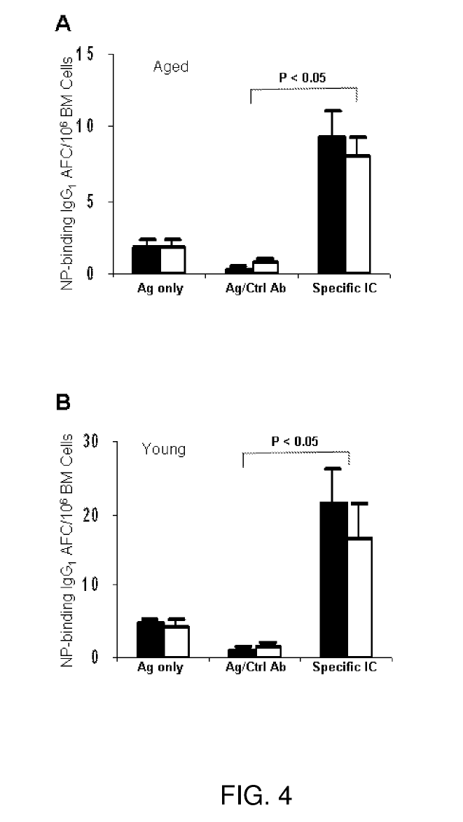

The frequencies of IgG1 AFCs in the bone marrow (BM) by ELISPOT assay at 12 days after secondary immunization was examined as previously described (Han et al., 2003; Zheng et al., 2007). Long-lived BM AFCs are derived from GC reaction and responsible for the long-term maintenance of antibody titers (Paramithiotis and Cooper, 1997; Slifka et al., 1995; Manz et al., 2005; Manz et al., 1997). Our results showed that the sizes of the BM AFC pool in both young and aged mice were significantly increased by IC immunization compared to immunization with antigen alone or antigen with control antibody (FIG. 4) (Zheng, 2007). The ratios of high affinity AFCs (NP₅-binding) to total AFCs (NP₂₅-binding) are commonly used as an index for affinity maturation (Han et al., 2003; Han et al., 2004; Takahashi et al., 1999; DiLillo et al., 2008). IC immunization also significantly increased NP₅-binding high affinity AFCs, demonstrating an effective selection process in the mice immunized with ICs. Consistent with an enhanced functional GC reaction, immunization with ICs significantly increased the long-term BM AFCs in both young and aged mice (FIG. 4). The results show that the size of the BM IgG₁ NP-specific AFC pool in aged mice immunized with ICs was significantly larger than that in aged mice receiving antigen alone or antigen with control antibody (FIG. 4A). The ratio of high-affinity (NP₅-binding)/total (NP₂₅-binding) AFCs can be used as an index for affinity maturation.

In addition, like BM AFCs from young mice (FIG. 4B), most of the BM AFCs from aged mice were high affinity (NP5-binding) antibody producers (FIG. 4A). These results demonstrate effective selection and enrichment of high-affinity AFCs in aged mice immunized with ICs.

### Example 5

### Immune Complex Immunization Enhances T Cell Priming In Vivo

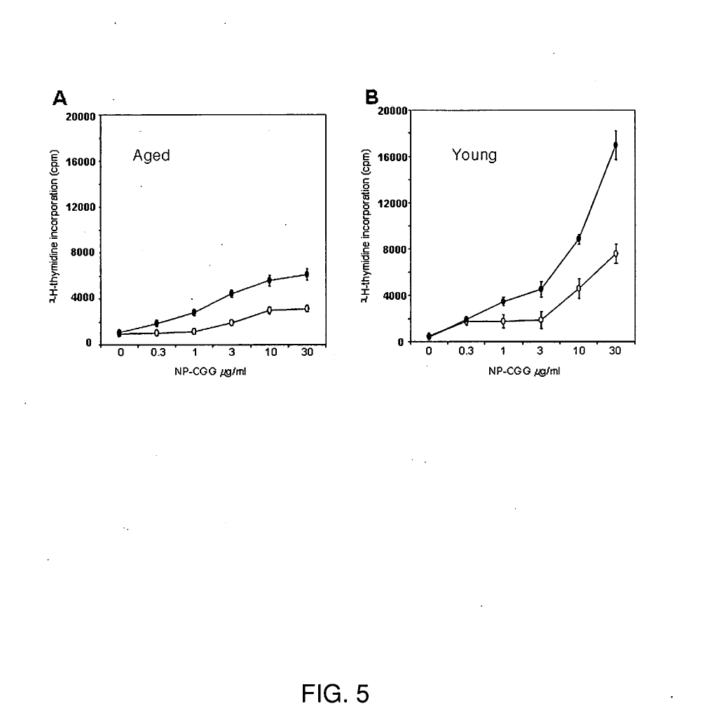

It has been shown that IC engagement of FcγRs induces DC activation and maturation (Regnault et al., 1999; Schuurhuis et al., 2002), and FcγR-mediated internalization of ICs by DCs is associated with enhanced presentation of both MHC class I- and II-restricted peptides (Regnault et al., 1999; Amigorena and Bonnerot, 1999a; Amigorena and Bonnerot, 1999b). To study the mechanisms responsible for the restoration of GC response by IC immunization in aged mice, the effect of ICs on T cell priming in vivo was evaluated, which will be the combined outcome of DC maturation, antigen-presentation, and T cell activation. In the this study, antigen-specific recall proliferation was compared between draining LN cells from mice immunized with NP-CGG ICs and mice injected with NP-CGG with control antibody. Seven days after immunization, draining LN cells were cultured in the presence of various concentrations NP-CGG for 4 days. Cellular proliferation was measured by ³H-thymidine incorporation for the last 18 hours of culture. FIG. 5 shows that in vivo priming with ICs significantly enhanced the in vitro antigen-specific proliferation of draining LN cells. These findings indicate that one of the mechanisms underlying the enhancement of GC response in aged mice by IC immunization is through promoting T helper cell function.

### Example 6

### Exemplary Materials and Methods for Examples 7-11

The present example provides exemplary materials and methods to practice the invention.

### Animals

Young (2-4 month-old) and aged (20-24 month-old) Balb/c (H-2^{d}) mice were from the Charles River Laboratory (Wilmington, Mass.) from cohorts maintained by the National Institute on Aging, NIH. Animal experimentation was performed in accordance with protocols approved by IACUC of Baylor College of Medicine.

### Influenza Virus, Vaccines and Immunization

Positive controls were young or aged mice immunized with ten 50% minimum infectious doses (MID₅₀) live mouse-adapted influenza A/Taiwan/1/86 (H1N1) intranasally (i.n.). Purified formalin-inactivated monovalent influenza A/Taiwan/1/86 (Connaught Laboratories, Swiftwater, Pa.) were used to immunized mice alone, or to form ICs with anti-NP mouse mAb (clone AA5H, IgG2a, Serotec, Raleigh, N.C.) or with isotype control mAb (clone C1, IgG₂ₐ). The dosage was the amount of inactivated vaccine containing 5 μg HA/mouse. The amount of anti-NP or control mAb was 5 μg/mouse. The ICs (or vaccine plus control mAb) was prepared by incubating equal amount of antigen and mAbs at 37° C. for 2 hours, then at 4° C. for 18 hours. Although NP is an internal protein, one can detect NP in our vaccine preparation by anti-nucleoprotein mAb (ELISA). The HA/NP ratio in exposed surface of the vaccine is about 2,000/1 (unpublished data). Mice were immunized 200 μl/mouse intraperitoneally (i.p). In some experiments, a second injection was given 5 weeks later.

CTL assay

Influenza-specific CTL activity was measured as was described earlier (Mbawuike et al., 1996). Briefly, 12 days after boost, spleen cells were prepared and stimulated for 6 days with virus-infected syngeneic spleen cells, or medium only. Cells were then washed and titrated in the specific cytotoxicity assay. Target cells were P815 (H-2 d) cells infected with live virus, P815 cells exposed to medium only, or EL-4 (H-2 b) cells infected with virus. Cytotoxicity was determined after 4 h by measuring released ⁵¹Cr. Specific CTL activity will be calculated as: (experimental release spontaneous release)/(maximum release spontaneous release)×100%.

### Flow Cytometry

For measurement of intracellular production of individual cytokines (IL-4, IL-10, and INF-γ), cultured cells were stimulated with 50 ng/ml PMA and 500 ng/ml ionomycin for 1 hour and with 10 μg/ml Brefeldin A (all from Sigma) for additional 4 hours. Cells then were washed and stained with FITC- or biotin labeled antibodies to CD3, CD4, and CD8, followed by streptavidin-TC. Cells were then washed and fixed with 4% paraformaldehyde at room temperature for 10 minutes. Cells were then treated with 0.5% saponin at room temperature for 10 minutes. Finally, cells were washed and incubated with PE-labeled anti-INF-7 antibody. Dendritic cells were stained with anti-CD11c-APC, anti-CD86-PE, and anti-1-A/I-E-biotin, followed by streptavidin-PerCP. All staining reagents were from BD/Pharmingen. Samples were collected on a FACScan machine (Becton Dickinson; Mountain View, Calif.) and analyzed using Flow Jo software (Tree Star Inc., San Carlos, Calif.).

### Detection of Influenza-Specific Antibodies by ELISA

Influenza HA- or NP-specific antibodies in mouse sera were determined by ELISA as described (Mbawuike et al., 1999). Briefly, microplates were coated with HA- or NP overnight and then blocked with 10% FCS. Samples were added and incubated for 1 hour at 37° C. and washed. HRP-conjugated goat anti-mouse IgG1, IgG₂ₐ, and IgM (Southern Biotechnology Associates, Birmingham, Ala.) were used as secondary detection reagents. Levels of HA- or NP specific antibodies were calculated using standard sera or mAb to NP.

### Measurement of Antibody-Forming Cells (AFCs) by ELISPOT Assay

The frequencies of specific AFCs from both splenocytes and bone marrow (BM) cells were estimated by ELISPOT assay as described (Zheng et al., 2002; Han et al., 2003). Briefly, nitrocellulose filters were coated with 5 μg/ml HA or NP in PBS at 4° C. overnight, and then blocked with 10% FCS in PBS. Splenocytes (5×10⁵ cells/well) or BM cells (10⁶ cells/well) were incubated on the filters in 96-well plates at 37° C., 5% CO₂. After 2-hour incubation, filters were washed with PBS containing 50 mM EDTA once, followed by PBS containing 0.1% Tween 20 twice and PBS once. Filters were double-stained with AP-conjugated anti-mouse IgM and HRP-conjugated anti-mouse IgG1 antibodies. AP and HRP activities were visualized using AEC and napthol AS-MX phosphate/Fast Blue BB, respectively.

### Dendritic Cell Isolation and Stimulation

Splenic DCs were labeled by incubating with anti-CD11c-biotin followed by streptavidin-microbeads. DCs were positively isolated passing through a magnetic column twice. Procedures of MACS separation were according to manufacturer's instructions (Miltenyi Biotec, Gladbach, Germany). Purified DCs were incubated for 48 hours with immune complex vaccine or vaccine mixed with isotype control antibody.

### Statistical Analysis

Student's t test of unpaired data was used to determine the significance of differences in means. A value of p<0.05 was considered to be statistically significant.

### Example 7

### Immunization with Immune Complex Vaccines Enhances Influenza-Specific CTL Activity in Aged Mice

To investigate the role of IC in regulating cellular immunity, IC vaccination enhancement of CTL responses against viral infection, such as influenza infection, was examined. Both human and animal studies have shown that CD8⁺ CTL activity plays a major role in promoting recovery from severe influenza infection (Bender et al., 1992; McMichael et al., 1983). The prevalence of prolonged and more severe infections in the elderly may be due to diminished CTL responses (Mbawuike et al., 2007; Zhang et al., 2002; Mbawuike et al., 1996). Delayed development and reduced levels of CTL activity as well as decreased splenic CTL precursor cells have been observed in aged mice compared with young mice (Mbawuike et al., 2007; Zhang et al., 2002; Mbawuike et al., 1996).

It was investigated whether IC vaccination can repair the diminished specific CTL response to influenza A in aged mice by i.p. immunizing aged and young Balb/c (H-2^{d}) mice with ICs consisting of inactivated monovalent influenza A/Taiwan/1/86 (H1N1) and anti-influenza A NP mAb (Zheng et al., 2007). Control groups include: (1) mice immunized with ten 50% minimum infectious doses (MID₅₀) live mouse-adapted influenza A/Taiwan/1/86 i.n.; (2) mice i.p. immunized with inactivated monovalent influenza A/Taiwan/1/86 only; and (3) mice i.p. immunized with inactivated monovalent influenza A/Taiwan/1/86 plus isotype-matched control mAb.

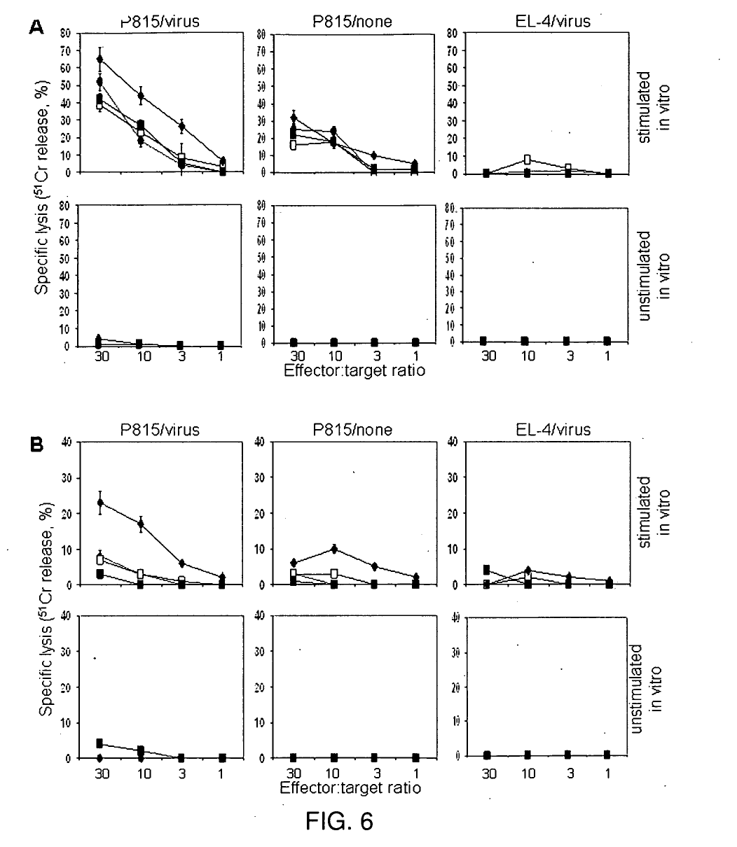

The results show that after in vitro stimulation with influenza A virus, effector cells generated from all groups of young mice, either immunized with different forms of inactivated vaccines or infected by live virus, exhibited significant virus-specific CTL activity (FIG. 6A). Interestingly, effector cells generated from young mice immunized with ICs showed higher CTL activity than those from mice immunized with other forms of vaccines including live-virus infection. In marked contrast, effector cells generated from aged mice, immunized either with inactivated vaccines (alone or with control mAb), or even infected with live influenza virus, exhibited little CTL activity (FIG. 6B). These results support earlier findings (Mbawuike et al., 1993; Powers and Belshe, 1993; Powers, 1993; Mbawuike et al., 1996; Zhang et al., 2000; Po et al., 2002; Mbawuike et al., 1997; Mbawuike et al., 1990) demonstrating age-related impairment in generating influenza virus-specific MHC class I-restricted CTL activity. Remarkably, effectors generated from aged mice immunized with ICs exhibited significantly enhanced virus-specific CTL activity (FIG. 6B). Therefore, the results demonstrated that immunization with ICs can significantly alleviate age-related deficiency in generating CTL response against influenza A virus.

### Example 8

### Immunization with IC Enhances IFN-γ Production by CD8⁺ Cells from Aged Mice

IFN-γ is a pivotal cytokine for the induction of anti-viral CTL responses. It has been shown that there is a strong correlation between CD8⁺ CTL activity and IFN-γ synthesis (Bender et al., 1991; Di Fabio et al., 1994; Taylor et al., 1985). In earlier work, there was a significant reduction of IFN-γ production by CD8⁺ T cells responding to influenza virus in aged mice (Mbawuike et al., 1996; Zhang et al., 2000).

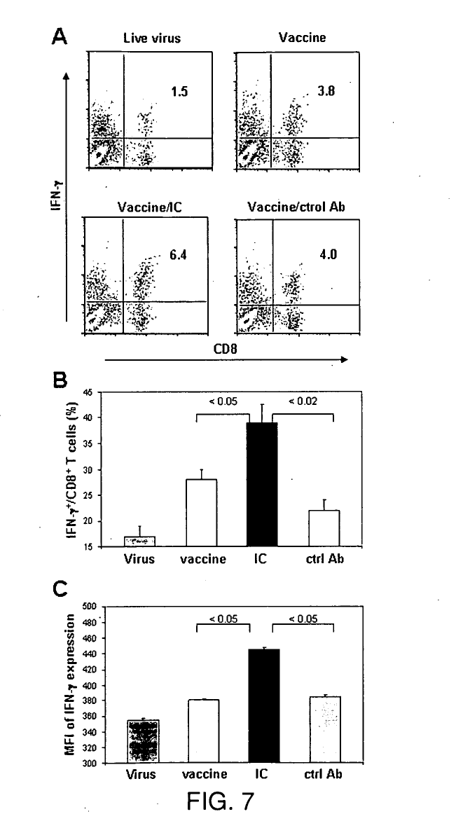

To determine whether IC vaccination enhances IFN-γ production by influenza A-specific cytotoxic CD8⁺ cells from aged mice, the levels of intracellular IFN-γ were measured in CD8⁺ cells. Splenic cells from aged mice immunized with different vaccines (live influenza A virus, inactivated vaccine alone, ICs containing inactivated vaccine and specific mAb, or inactivated vaccine plus control mAb) were stimulated with virus-infected stimulator cells for 4 days and examined for IFN-γ production by intracellular cytokine staining. The results show that the levels of IFN-γ-producing CD8⁺ cells were significantly higher in cultures from IC immunized aged mice than those from other immunization groups (FIGS. 7A and 7B). Importantly, CD8⁺ T cells from IC-immunized aged mice also make more IFN-γ per cell since the mean fluorescence intensity (MFI) of IFN-γ staining in CD8⁺ T cells from IC-immunized group was significantly higher than that of other groups (FIG. 7C).

### Example 9

### Immunization with Anti-NP IC Vaccine Enhances Antibody Response to Both NP and HA in Aged Mice

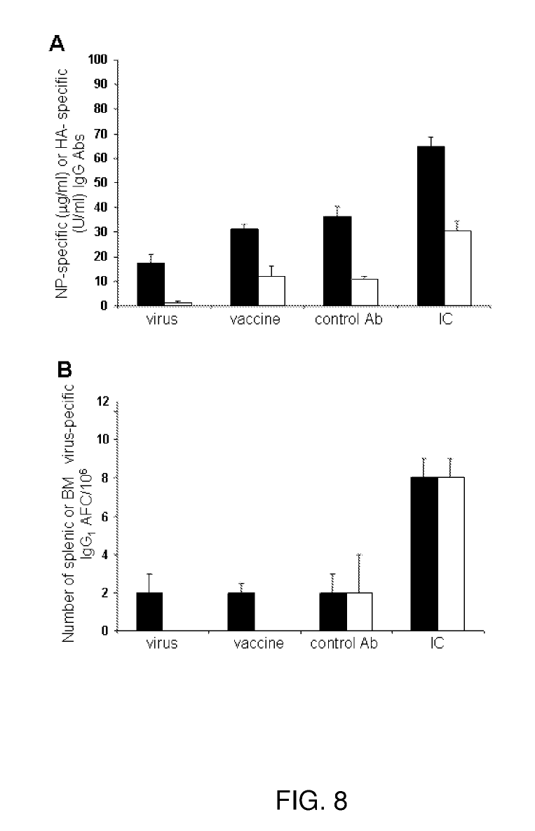

Another indicator of protection to influenza A infection is the Antibody titers of haemagglutination inhibition (HI) (Potter and Oxford, 1979; Pereira et al., 1972; Goodeve et al., 1983). There is a correlation between low HI serum antibodies and low efficacy of influenza vaccines in the elderly (Strassburg et al., 1986; Gross et al., 1995; Keren et al., 1988; Phair et al., 1978; Arden et al., 1986; Barker and Mullooly, 1986). To test if IC immunization can enhance antibody responses to both NP and HA in aged mice, the levels were determined of serum antibodies and numbers of antibody-forming cells (AFCs) to NP and HA 16 days after immunization. IC vaccine has a significant enhancing effect on antibody levels specific for both NP and HA in aged mice (FIG. 8A). Consistently, the numbers of virus-specific AFCs in the spleen and bone marrow (BM) were significantly increased in aged mice receiving vaccine/anti-NP IC compared to those in mice receiving other forms of vaccines (FIG. 8B). Thus, the results demonstrate that IC vaccination can repair the age-related deficiency in both cellular and humoral immunity to influenza A virus.

### Example 10

### In Vivo Priming with IC Enhances Type 1 Cytokine—But not Type 2 Cytokine Production by both CD4 and CD8 T Cells in Aged Mice

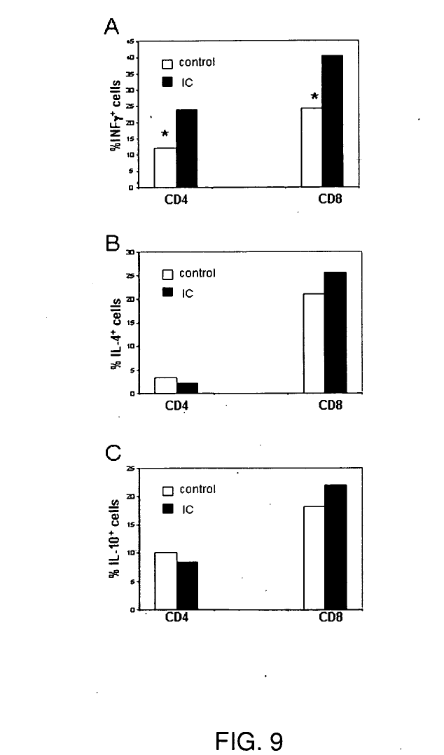

To determine the effect of IC immunization on cytokine production and Th1/Th2 responses, the cytokine profiles were investigated of both CD4⁺ and CD8⁺ T cells after in vivo priming with IC or control influenza A vaccine. One week after immunization, draining lymph node (LN) cells were stimulated with the immunizing influenza vaccine and antigen-specific cytokine production was measured by intracellular cytokine staining. FIG. 9A shows that aged mice immunized with IC generated higher frequencies of IFN-γ-producing CD4⁺ and CD8⁺ T cells than control mice immunized with vaccine and control antibody. However, there was no difference in the frequency of cells producing IL-4 or IL-10 between IC-immunized mice and control animals (FIGS. 9B and 9C). These findings indicate that in vivo priming with IC predominantly promotes Th1 response and has less effect on Th2 response.

### Example 11

### Immune Complex Promotes Maturation and Function of Dendritic Cells from Aged Mice

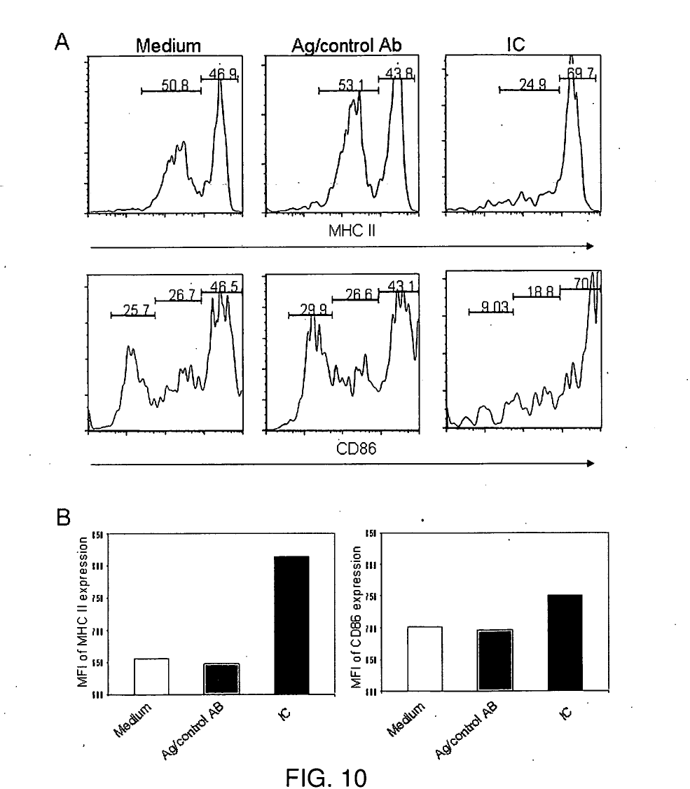

To further determine the mechanisms underlying the immune enhancing effects of IC vaccine in aged mice, the effects of ICs in promoting DC maturation and function were investigated. Splenic DCs were isolated and incubated with IC vaccine or vaccine/control antibody for two days. The expression levels of MHC class II and costimulatory molecule CD86 were evaluated by flow cytometry. The results show that the frequencies of DCs with higher expression level of MHC class II molecule or CD86 were significantly increased in DCs cultures with ICs compared to that in DC cultures with vaccine plus control antibody (FIG. 10A). In addition, the overall expression of MHC class II and CD86 was significantly up-regulated on DCs stimulated with IC vaccine (FIG. 10B). Thus, these findings demonstrate that in specific embodiments enhanced DC maturation and function contribute to the mechanisms that improve immune responses to influenza A by IC vaccination.

### Example 12

### Significance of the Present Invention

The present invention demonstrates that at least age-associated impairment in GC reaction can be significantly restored by IC immunization. This improvement of GC formation, coupled with preserved clonal competition and selection, in specific embodiments, results in an overall improvement of antibody affinity maturation as well as an enriched pool of memory B cells and long-lived BM plasma cells in aged animals.

It is generally believed that the humoral responses in the aged are overall diminished. However, the mechanisms responsible for this age-related immune deficiency are not well understood. The evolution of an antibody response can be roughly divided into three phases: the initial pre-GC extrafollicular antibody response, GC response and post-GC differentiation pathway. Currently available evidence suggests that a diminished GC response is the main cause for the impaired antibody responses in aging (Kosco et al., 1989; Zheng et al., 1997; Han et al., 2003; Miller and Kelsoe, 1995; Lu and Cerny, 2002), whereas the pre- and post GC pathways are largely functional except that aged BM is less supportive for long-term AFC generated during a primary antibody response (Zheng et al., 1997; Han et al., 2003). It has been suggested that age-related dysfunctions in Th cells and follicular dendritic cells (FDC) play critical roles in GC deficiency in aging (Chakravarti and Abraham, 1999; Miller, 1995; Szakal et al., 1990; Szakal et al., 1992). Thus, the GC response in aging may be improved by either circumventing the requirements for Th and/or FDC, or overcoming deficiencies in Th and/or FDC.

Several aspects of the invention account for the significantly improved GC response in aged mice by IC immunization. In one embodiment, ICs play an important role in modulating the antibody responses by regulating FDC functions (Qin et al., 2000; Aydar et al., 2002). It has been shown that the impaired GC formation and Ig hypermutation in athymic mice with limited numbers of T cells were restored by administrating antibodies specific for the immunizing antigen (Song et al., 1999). Similarly, immunization with preformed IC enhanced Ig hypermutation and alters the process of clonal competition (Nie et al., 1997). The BCR affinity threshold for antigen uptake and presentation is significantly lowered by oligomerization of antigens with antibodies (Batista and Neuberger, 1998). The ICs may increase the avidity of antigen-BCR interaction and enhance the BCR-mediated signals, in certain aspects of the invention. Additionally, by fixing complement and bridging BCR with complement, the ICs elicit co-stimulatory signals through co-receptors such as CD19 (Tsoko et al., 1990; Carter and Fearon, 1992; Dempsey et al., 1996). Finally, in other embodiments other factors may also contribute to the improved GC response induced by IC immunization in aged mice, including improved DC maturation and activation, enhanced antigen presentation, and increased Th cell activation. All these factors, in certain aspects of the invention, may exert their effects independently or synergistically, resulting in an overall enhanced GC response. Therefore, the results indicate that impaired GC responses in aging can be overcome by IC immunization, which has important implications in designing vaccine compositions and immunization protocols for the elderly population and certain T-cell deficient patients, such as AIDS.

The present invention also demonstrates that age-associated impairment in generating functional influenza-specific CTLs can significantly be alleviated by immunization with immune complex vaccine. This improved CTL function, together with enhanced virus-specific humoral immunity, results in an overall improved influenza-specific immune response in aged animals.

Complex changes in the immune response occur as species including mouse and man undergo post-maturational aging. The most profound changes in an aged immune system are in the T cell compartment (Chakravarti and Abraham, 1999; Hodes and Fauci, 1996; Cossarizza et al., 1997; Miller, 1995). The age-related decline in T cell function results in a shift in the phenotype of circulating CD4⁺ T cells, with a decrease in naive CD4⁺ T cells and relative accumulation of memory CD4⁺ T cells. In addition, the memory T cells include a spectrum of normal functioning and hypofunctioning T cells, compared with memory T cells in young controls. The decrease in functioning cells results in impaired proliferating capacity and impaired expression of IL-2/IL-2 receptor (Miller, 1995). Although less numerous, studies on CD8⁺ T cells have also found some age-related changes (Bums and Goodwin, 1997; Hirokawa, 1998). It has been demonstrated that influenza virus-specific class I-restricted CD8⁺ CTL activity was significantly diminished in elderly persons (Mbawuike et al., 1993; Powers and Belshe, 1993; Powers, 1993) as well as in aged mice (Mbawuike et al., 1996; Zhang et al., 2000; Po et al., 2002). Existing evidence indicates that there are age-related changes in DCs. It has been shown that although the elderly are able to generate large numbers of DCs from PBMC and that these cells have a phenotype and antigen presentation capacity similar to those of DCs from young controls, DCs in the elderly may have an impaired capacity to cross tissue barriers and to trigger cytokine production from specific T cells (Wick and Grubeck-Loebenstein, 1997). Therefore, the ability of DCs to differentiate after interaction with T cells is impaired with aging, in certain aspects of the invention, and this circumstance is related to the observation that production of GM-CSF, a key DC growth factor, was diminished in the elderly (Pawelec et al., 1999). Thus, improvement of DC maturation and function is useful in overcoming age-related impairment in immunity, in specific aspects of the invention.

Immune cells express four types of FcγR, FcγRI, IIB, III and IV (Ravetch and Bolland, 2001; Takai, 2002; Nimmerjahn and Ravetch, 2006). Binding of FcγR can lead to either activating or inhibitory signaling depending on which specific FcγR being engaged. Activating FcRs (FcγRI, III, and IV) associate with the immunoreceptor tyrosine-based activation motif (ITAM)-containing γ-chain and their engagement results in src and syk kinase-mediated activation. In contrast, the inhibitory FcR (FcγRIIB) is a receptor containing a cytoplasmic immunoreceptor tyrosine-based inhibition motif (ITIM) that inhibits ITAM-mediated signals through the recruitment of the inositol-phosphatase SHIP (Bolland et al., 1998; Pearse et al., 1999). Balanced signaling through activating and inhibitory FcR intimately regulates the activity of various cells in the immune system (Ravetch and Bolland, 2001; Takai, 2002; Takai et al., 1994; Takai et al., 1996). Recent work has demonstrated that different subclasses of IgG have differential affinities for specific activating FcγRs compared with their affinities for the inhibitory FcγR, leading to substantial differences in their ability to mediate effector functions (Nimmerjahn and Ravetch, 2006; Nimmerjahn and Ravetch, 2005). An A/I (activating-to-inhibitory) ratio can be used to describe this differential affinity for functional distinct FcRs by a specific IgG subclass (Nimmerjahn and Ravetch, 1995; Nimmerjahn et al., 2005). It has been shown that the hierarchy of in vivo biological activity for the IgG subclasses is IgG2a≧IgG2b>IgG1>>IgG3, mirroring the hierarchy based on the A/I ratios (Nimmerjahn and Ravetch, 2006 Nimmerjahn and Ravetch, 2005).

In embodiments of the present invention, an IgG2a monoclonal antibody specific for NP of influenza A virus was used to form immune complexes. The data demonstrated that when aged mice were immunized with this IgG2a immune complex, the influenza-specific immunity was significantly improved compared to that in aged mice received other forms of vaccines, including the same vaccine plus an isotype control antibody. In particular, immune complex vaccination in aged mice induced a significant virus-specific CTL response that was almost undetectable in aged mice immunized with other forms of vaccination, including live-virus infection. Both viral specific cytotoxicity and INF-γ production by CD8⁺ T cells were significantly enhanced in aged animals immunized with immune complex vaccine. In addition, antibody responses against both surface antigen (HA) and core antigen (NP) were improved by immune complex immunization. In vivo priming experiments showed that the IgG2a immune complex predominantly promotes type 1 cytokine production in both CD4⁺ and CD8⁺ T cells, suggesting that both Th1 and cytotoxic T cell functions are improved by immune complex vaccination. Potential mechanisms underlying the immune enhancing effect by immune complex vaccines were investigated, and the results have shown that, when stimulated with immune complex vaccine, DCs from aged mice expressed significantly higher levels of MHC class II and costimulatory molecules, indicating that immune complexes can enhance the maturation and function of aged DCs. Thus, the findings indicate that at least impaired anti-viral responses in aging can be significantly improved by immunization with immune complex vaccines, which is useful for designing vaccine compositions and immunization protocols for the elderly population and certain T-cell deficient patients, such as AIDS.

### Example 13

### Enhanced GC Reaction after Immunization with IC Containing HIV-1 and Anti-HIV Antibody

The success of the previous examples in modulating age-associated immune deficiency and in enhancing overall antibody responses in both young and aged mice by ICs inspired the evaluation of the effect of IC immunization in eliciting humoral responses to HIV-1. BALB/c mice (female, 10-weeks old) were immunized with ICs consisting of inactivated HIV-1 (strain 97ZA012, clade C, R5-dependent) and mouse anti-gp120 mAb (clone YZ23, IgG2a/κ) which is a neutralizing antibody specific for the gp120 superantigenic (SAg) epitope at residues 421-433 (Paulo et al., 2003). Control immunogen was prepared with inactivated virus and an isotype matched mAb (clone AA5H, anti-influenza A nucleoprotein, IgG2a/κ; Serotec, Raleigh, N.C.). The IC (or Ag/control Ab) was prepared by incubating the virus with mAb in a volume of 100 μl at 37° C. for 2 hours, then at 4° C. for 18 hours. Each mouse received a subcutaneous (s.c.) injection containing 1×10⁸ virions and 500 ng mAb at the base of tail.

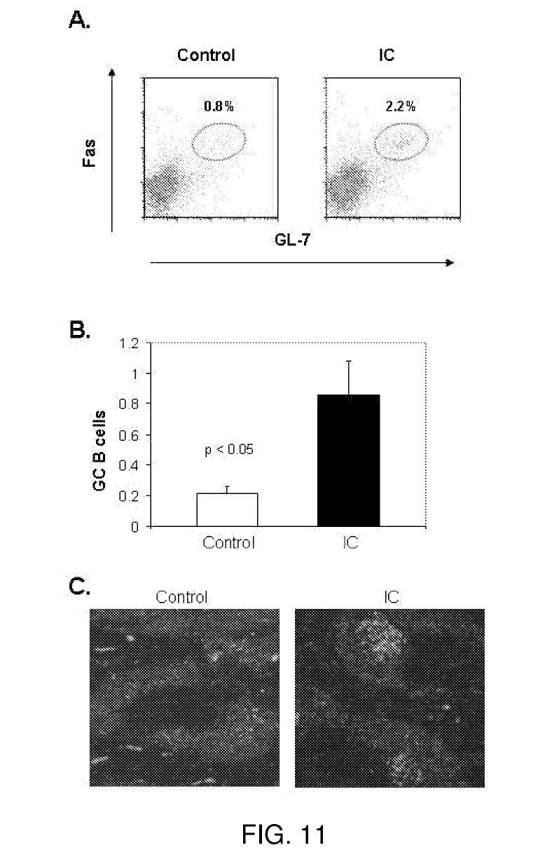

Twelve days after primary immunization, LNs and spleens were harvested for analysis. GC formation was evaluated as previously described (Han et al., 2003; Zheng et al., 2007; Han et al., 2004). The results showed that GC response in mice immunized with IC was significantly enhanced compared to that in mice immunized with Ag/control Ab (FIG. 11).

### Example 14

### Immune Complex Immunization Increases GP120-Specific Antibody Production and AFC Generation

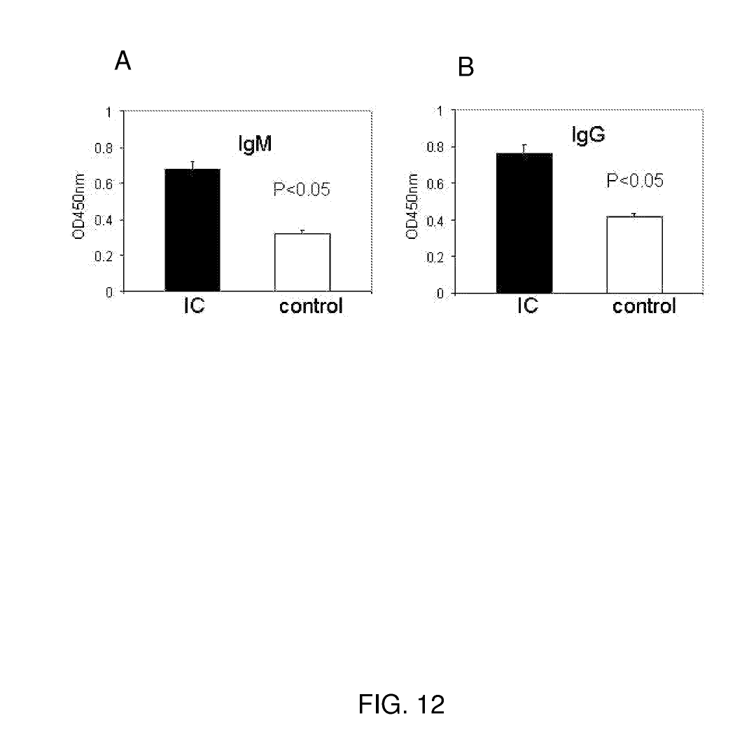

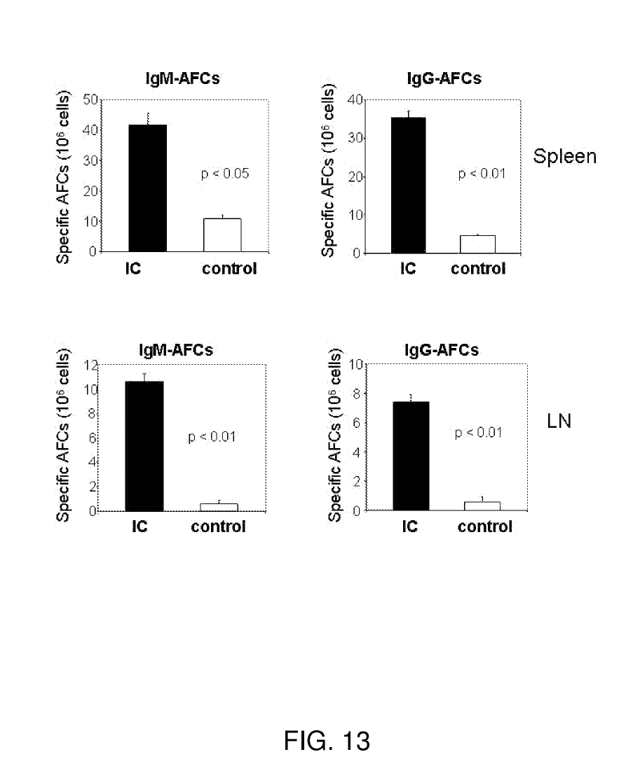

The effects of IC immunization on Ab production and the generation of AFCs was further evaluated. Consistent with the enhanced GC reaction, a significant increase in both IgM and IgG Abs to gp120 in mice immunized with ICs was observed (FIG. 12). In addition, IC immunization also resulted in enhanced IgM- and IgG-producing plasma cell responses to gp120 in the lymph nodes and spleens (FIG. 13). The frequencies of both IgM- and IgG-AFCs were increased significantly in the mice immunized with IC compared to controls. These findings demonstrated that IC immunization significantly promotes humoral responses against HIV-1 gp120.

### Example 15

### Immune Complex Immunization Enhances T-Cell Priming to HIV-1 In Vivo

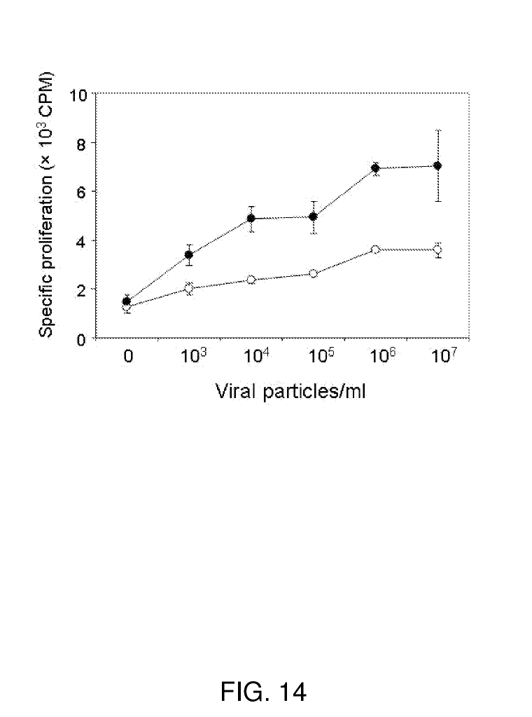

The finding that IC immunization improves antibody responses to HIV-1 prompted the evaluation of the effect of IC on T cell priming. Ag-specific recall proliferation was compared between draining LN cells from the mice immunized with ICs (containing inactivated HIV-1 (97ZA012) and anti-gp120) or control mix (inactivated HIV-1 mixed with isotype control antibody). At 12 days after immunization, draining LN cells were cultured in the presence of various concentrations of inactivated HIV virus for 3 days. Cellular proliferation was measured by ³H-thymidine incorporation for the last 18 hours of culture. The results showed that in vivo priming with IC significantly enhanced the in vitro Ag-specific proliferation of draining LN cells (FIG. 14) indicating that IC exert a potent positive effect on T cell responses to HIV-1.

### Example 16

### IC Immunization Promotes Memory AB Responses to HIV-1

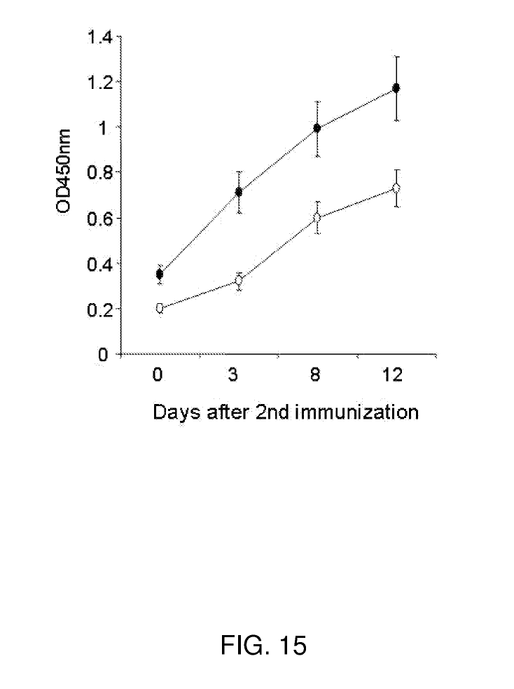

One embodiment for the criteria for a successful vaccine is that it should induce an effective memory response. To examine whether IC enhances the memory response to HIV, at 60 days after primary immunization, mice were given a second injection as in primary immunization as described above. Serum samples were collected at various days of secondary immunization. IgG Abs specific for gp120 were measured by ELISA. The data showed that anti-gp120 Ab production was significantly increased in mice immunized with ICs compared to that in control mice (FIG. 15).

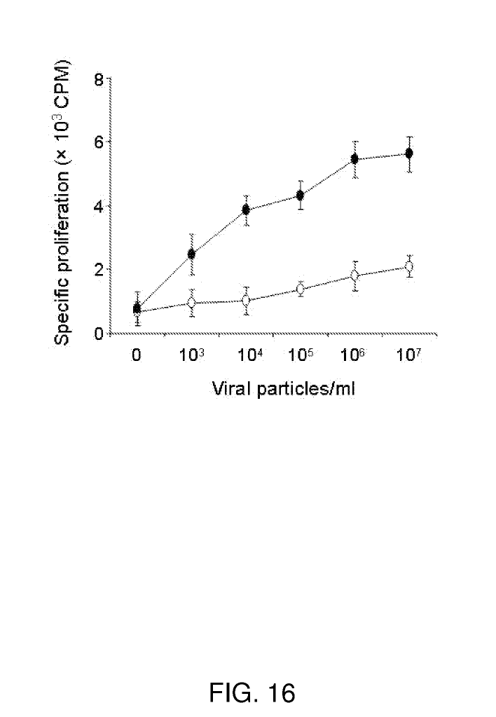

Additionally, B-cell recall proliferation was investigated following secondary immunization. Spleens were removed 12 days after secondary immunization. B cells were purified by MACS (via negative selection). Briefly, splenic single cell suspensions were incubated with biotinylated mAbs specific for CD4, CD8, Thy-1, Mac-1, Gr-1, and Ter-119 (all were purchased from PharMingen), and labeled T cells, macrophages, dendritic cells, neutrophils and erythroid cells were removed by incubating with streptavidin-microbeads (Miltenyi Biotec), and passing through a magnetic column. The purity of B-cells was >98%. The purified B-cells (2×10⁵/well) were cultured in the presence of various concentration of virus for 3 days. Cellular proliferation was measured by ³H-thymidine incorporation for the last 18 hours of culture. The results showed that B-cells from the mice immunized with IC proliferated more vigorously than the B cells from the control mice (FIG. 16), indicating that IC immunization generated a significantly more effective B-cell memory compartment compared to the controls. The result shown in FIG. 16 further confirms the findings that mice immunized with IC generated a more vigorous memory Ab response to HIV-1 than the controls.

### Example 17

### Immune Complex Immunization Elicits Neutralizing Antibodies to HIV-1

One embodiment of an effective antibody response to HIV-1 infection is to induce neutralizing antibodies. To examine whether IC immunization is able to promote production of neutralizing antibodies, the sera from mice immunized with IC or control Ab/Ag mix was analyzed for their neutralizing activity. The peripheral blood mononuclear cell (PBMC)-based neutralization assay was performed essentially as described previously (D'Souza et al., 1995; Mascola et al., 1996), but with p24 expression quantified using a Beckman Coulter HIV-1 p24 Antigen Assay Research Component Kit, giving a linear range of 50-3,200 μg/mL. Briefly, HIV-1 neutralization assays employ pooled PBMCs from normal blood donors prepared from buffy coats so that a series of experiments can be performed using the same PBMC pool. The cells were stimulated for 24 hours with PHA-P. Sera were diluted and transferred to the 96-well assay plate in quadruplicate. Virus stocks were diluted based on prior titrations to give approximately 100 TCID50/well, and mixed with equal volume of diluted sera. Virus and serum samples were incubated together for one hour at 37° in 5% CO₂. Then PHA-activated PBMC (3×10⁵/well) were added into the plates and incubated for 72 hours. Residual virus/antibody inoculum were removed by centrifuging and washing the plates three times with fresh medium. After an additional 24 hours incubation, 1% Triton-X was added to each well and p24 quantified by EIA (Coulter). The data were fitted with a sigmoidal dose-response curve with variable slope (Hill-type function) using Prism software (Moulard et al., 2002). The endpoint titer for neutralization is defined as the interpolated titer at which there is a 50% or a 90% inhibition of p24 expression relative to controls.

The results showed that the sera from mice immunized with immune complex exhibited about 10-fold higher neutralization titers compared to the controls at IC₅₀ (50% neutralization) (FIG. 16A). The IC immunization induced serum samples displayed titration-dependent neutralization (FIG. 16B). By contrast, IC₅₀ values for the control samples were not only very low but also no titration-dependent neutralization. This result indicates that IC immunization induced significantly higher neutralizing antibodies. Although the numbers of serum samples were small, the result was very promising.

### Example 18

### Immune Complex Immunization Promotes GC Reaction and Anti-GP120 AB Responses in CD4-Deficient Mice

Since the T helper function is usually severely impaired in AIDS patients, it investigated whether IC immunization can enhance Ab responses in the mice with diminished T helper function. It has previously been shown that CD4^{-/-} mice exhibit severely reduced T helper function and impaired B-cell response (Zheng et al., 2002). Therefore, CD4-deficient mice are an excellent model to evaluate whether diminished T help function can be overcome by IC immunization.

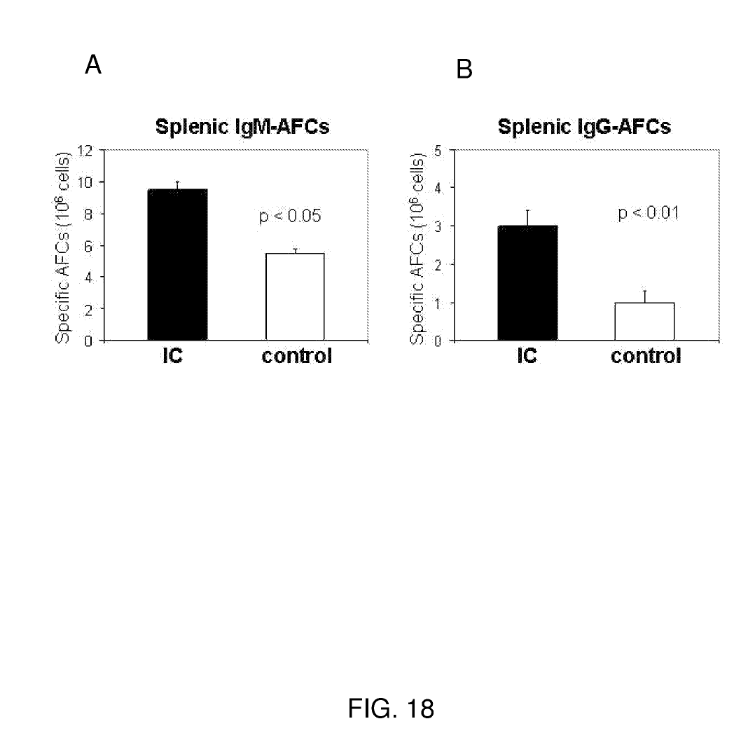

CD4-deficient mice were immunized with gp120-mAb IC or control immunogen as described above. Impressively, at 12 days after primary immunization, the levels of gp120-specific AFCs were significantly elevated in the mice immunized by IC compared to controls (FIG. 18). Thus, the data indicated that IC immunization can indeed be an effective approach to mitigate depressed T help function in a mouse model and improve humoral response to HIV-1.

### REFERENCES

All patents, patent applications, and publications mentioned in the specifications are indicative of the levels of those skilled in the art to which the invention pertains. All patents, patent applications, and publications are herein incorporated by reference to the same extent as if each individual publication was specifically and individually indicated to be incorporated by reference.

### Patents and Patent Applications

U.S. Pat. No. 4,196,265
U.S. Pat. No. 5,021,236
U.S. Pat. No. 3,817,837
U.S. Pat. No. 4,472,509
U.S. Pat. No. 4,938,948
U.S. Pat. No. 5,466,468
U.S. Pat. No. 4,938,948
U.S. Pat. No. 4,472,509
U.S. Pat. No. 3,850,752
U.S. Pat. No. 3,939,350
U.S. Pat. No. 4,277,437
U.S. Pat. No. 3,996,345
U.S. Pat. No. 4,275,149
U.S. Pat. No. 4,366,241
U.S. Pat. No. 4,938,948

### Publications

Allen, D., T. Simon, F. Sablitsky, K. Rajewsky, A. Cumano. Antibody engineering for the analysis of affinity maturation of an anti-hapten response. EMBO J. 7 (1988) 1995-2001.
Amigorena, S., C. Bonnerot. Fc receptor signaling and trafficking: a connection for antigen processing. Immunol. Rev. 172 (1999) 279-284.
Amigorena, S., C. Bonnerot. Fc receptors for IgG and antigen presentation on MHC class I and class II molecules. Semin. Immunol. 11 (1999) 385-390.
Arden, N. H., P. A. Patriarca, K. J. Lui, M. W. Harmon, F. Brandon, and A. P. Kendal. 1986. Safety and immunogenicity of a 45-microgram supplemental dose of inactivated split-virus influenza B vaccine in the elderly. J. Infect. Dis. 153:805-806.
Aydar, Y., P. Balogh, J. G. Tew, A. K. Szakal. Age-related depression of FDC accessory functions and CD21 ligand-mediated repair of co-stimulation. Eur. J. Immunol. 32 (2002) 2817-2826.
Barker, W. H., and J. P. Mullooly. 1986. Effectiveness of inactivated influenza vaccine among non-institutionalised elderly persons. In: A. P. Kendal and P. A. Patriarca, Editors, Options for the Control of Influenza, Alan R. Liss, New York, N.Y., pp. 169-182.
Batista, F. D., M. S. Neuberger. Affinity dependence of the B cell response to antigen: a threshold, a ceiling, and the importance of off-rate. Immunity 8 (1998) 751-759.
Bender, B. S., M. P P. Johnson, and P. A. Small. 1991. Influenza in senescent mice: impaired cytotoxic T-lymphocyte activity is correlated with prolonged infection. Immunology 72:514-519.
Bender, B. S., T. Croghan, L. Zhang, and P. A. Small. 1992. Transgenic mice lacking class I major histocompatibility complex-restricted T cells have delayed viral clearance and increased mortality after influenza virus challenge. J. Exp. Med. 175:1143-1145.
Benner, R., R. Meima, G. M. van der Meulen, W. B. Muiswinkel. Antibody formation in mouse bone marrow. I. Evidence for the development of plague-forming cells in situ. Immunology 26 (1974) 247-255.
Benner, R., W. Haaijman, J. Haaijman. The bone marrow: the major source of serum immunoglobulins, but still a neglected site of antibody formation. Clin. Exp. Immunol. 46 (1981) 1-8.
Berek, C., A. Berger, and M. Apel. 1991. Maturation of the immune response in germinal centers. Cell 67:1121-1129.
Bolland, S., R. N. Pearse, T. Kurosaki, and J. V. Ravetch. 1998. SHIP modulates immune receptor responses by regulating membrane association of Btk. Immunity. 8:509-516.
Bothwell, A. L., M. Paskind, M. Reth, T. Imanishi-Kari, K. Rajewsky, D. Baltimore. Heavy chain variable region contribution to the NP^{b} family of antibodies: somatic mutation evident in a gamma 2a variable region. Cell 24 (1981) 625-637.
Burns, E. A., and S. Goodwin. 1997. Immunodeficiency of aging. Drugs. Aging. 11:374-397.
Carter, R. H., D. T. Fearon. CD19: lowing the threshold for antigen receptor stimulation of B lymphocytes. Science 256 (1992) 105-107.
Castel, S. C. 2000. Clinical relevance of age-related immune dysfunction. Clin. Infect. Dis. 31:578-585.
Chakravarti, B., and G. N. Abraham. 1999. Aging and T-cell mediated immunity. Mech. Ageing. Dev. 108:183-206.
Chakravarti, B., G. N. Abraham. Aging and T-cell-mediated immunity. Mech. Ageing Dev. 108 (1999)183-206.
Cossarizza, A., C. Ortolani, D. Monti, and C. Franceschi. 1997. Cytometric analysis of immunosenescence. Cytometry 27:297313.
Dal Porto, J. M., A. M. Haberman, M. J. Shlomchik, G. Kelsoe. Antigen drives very low affinity B cells to become plasmacytes and enter germinal centers. J. Immunol. 161 (1998) 5373-5381.
Dempsey, P. W., M. E. Allison, S. Akkaraju, C. C. Goodnow, D. T. Fearon. C3d of complement as a molecular adjuvant: bridging innate and acquired immunity. Science 271 (1996) 348-350.
Di Fabio, S., I. N. Mbawuike, H. Kiyono, K. Fujihashi, R. B. Couch, and J. R. McGhee. 1994. Quantitation of human influenza virus-specific cytotoxic T lymphocytes: correlation of cytotoxicity and increased numbers of IFN-γ producing CD8⁺ T cells. Int Immunol. 6:11-19.
DiLillo, D. J., Y. Hamaguchi, Y. Ueda, K. Yang, J. Uchida, K. M. Haas, G. Kelsoe, and T. F. Tedder. 2008. Maintenance of long-lived plasma cells and serological memory despite mature and memory B cell depletion during CD20 immunotherapy in mice. J Immunol 180:361-371.
Doria, G., D'Agostaro, G., Poretti, A. Age dependent variations of antibody avidity. Immunology 35 (1978) 601-6111.
D'Souza, M. P., G. Milman, J. A. Bradac, D. McPhee, C. V. Hanson, and R. M. Hendry. 1995. Neutralization of primary HIV-1 isolates by anti-envelope monoclonal antibodies. Aids 9:867-874.
Fukuda, F., C. B. Bridges, and T. L. Brammer. 1999. Prevention and control of influenza: recommendation of the Advisory Committee on Immunization Practices. MMWR Morb. Mortal. Wkly. Rep. 48:1-37.
Gavazzi, G., and K. H. Krause. 2002. Aging and infection. Lancet Inf. Dis. 2:259-666.
Ginaldi, I., M. De Martinis, A. D'Ostilio, L. Marini, M. F. Loreto, V. Martorelli, and D. Quaglino. 1999. Immunological changes in the elderly. Aging (Milano) 11:281-286.
Ginaldi, L., M. F. Loreto, M. P. Corsi, M. Modesti, and M. De Martinis. 2001. Immunosenescence and infectious diseases. Microbes Infect. 3:851-857.
Goodeve, A., C. W. Potter, A. Clark, R. Jennings, G. C. Schild, and R. Yetts. 1983. A graded dose study of inactivated surface antigen influenza B vaccine in volunteers. J. Hyg. 90:107-115.
Gotch, F., A. McMichael, G. Smith, and B. Moss. 1987. Identification of viral molecules recognized by influenza-specific human cytotoxic T lymphocytes. J. Exp. Med. 65:408-416.
Gross, P. A. A. W. Hermogenes, H. S. Sacks, J. Lau, and R. A. Levandowski. 1995. The efficacy of influenza vaccine in elderly persons: a meta-analysis and review of the literature. Ann Intern Med. 23:518-527.
Gross, P. A., A. W. Hermogenes, H. S. Sacks, J. Lau, and R. A. Levandowski. 1995. The efficacy of influenza vaccine in elderly persons: a meta-analysis and review of the literature. Ann. Intern. Med. 123:518-527.
Hamano Y., H. Arase, H. Saisho, T. Saito. Immune complex and Fc receptor-mediated augmentation of antigen presentation for in vivo Th cell responses. J. Immunol. 164 (2000) 6113-6119.
Han, S., K. Hathcock, B. Zheng, T. Kepler, R. Hodes, G. Kelsoe. Cellular interaction in germinal centers: roles of CD40 ligand and B7-2 in established germinal centers. J. Immunol. 155 (1995) 556-567.
Han, S., K. Yang, Z. Ozen, G. E. Marinova, W. Pen, Kelsoe, and B. Zheng. 2003. Enhanced differentiation of splenic plasma cells but diminished long-lived high-affinity bone marrow plasma cells in aged mice. J Immunol. 170:1267-1273.
Han, S., Marinova, E., Zheng, B. Rectification of age-related impairment in Ig gene hypermutation during a memory response. Int. Immunol. 16 (2004) 525-532.
Han, S., Yang, K., Ozen, Z., Peng, W., Marinova, E., Kelsoe, G., Zheng, B. Enhanced differentiation of splenic plasma cells but diminished long-lived high-affinity bone marrow plasma cells in aged mice. J. Immunol. 170 (2003) 1267-1273.
Hirokawa, K. 1998. Immunity and aging. In: Pathy MSJ, ed. Principles and practice of geriatric medicine. 3d ed. New York: John Wiley and Sons, p35-47.
Hodes, R. J., and A. S. Fauci, eds. Report of Task Force on Immunology and Aging. Bethesda, Md.: National Institutes of Aging and of Allergy and Infectious Diseases, US Department of Health and Human Services, March 1996.
Jacob, J., R. Kassir, G. Kelsoe. In situ studies of the primary response to (4-hydroxy-3-nitrophenyl)-acetyl. I. The architecture and dynamics of responding cell populations. J. Exp. Med. 173 (1991) 1165-1175.
Jacob, J., G. Kelsoe, K. Rajewsky, and U. Weiss. 1991. Intraclonal generation of antibody mutants in germinal centres. Nature 354:389-392.
Kelsoe, G. 1995. In situ studies of the germinal center reaction. Adv Immunol 60:267-288.
Kelsoe, G. 1995. The germinal center reaction. Immunol Today 16:324-326.
Keren, G., S. Segev, A. Morag, Z. Zakay-Rones, A. Barzilai, and E. Rubinstein. 1988. Failure of influenza vaccination in the aged. J. Med. Virol. 25:85-89.
Kishimoto, S., Tomino, S, Misuya, H. Fujiwara, Tsuda, H. Age-related decline in the in vitro and in vivo synthesis of anti-tetanus toxoid antibody in humans. J. Immunol. 125 (1980) 2347-2352.
Klaus, G. G. B., Humphrey, J. H. A re-evaluation of the role of C3 in B-cell activation. Immunol. Today 7 (1986) 163-171.
Kosco, M. H., Burton, G. F., Kapasi, Z. F. Szakal, A. K., Tew, J. G. Antibody-forming cell induction during an early phase of germinal center development and its delay with aging. Immunology 68 (1989) 312-318.
Liu, Y. J., and C. Arpin. 1997. Germinal center development. Immunol Rev 156:111-126.
Lu, Y. F, Cerny, J. Repertoire of antibody response in bone marrow and the memory response are differentially affected in aged mice. J. Immunol. 169 (2002) 4920-4927.
Lukacher, A. E., V. L. Braciale, and T. J. Braciale. 1984. In vivo effector function of influenza virus-specific cytotoxic T lymphocyte clones is highly specific. J. Exp. Med. 60:814-826.
M. Reth, G. Hammerling, K. Rajewsky. Analysis of the repertoire of anti-NP antibodies in C57BL/6 mice by cell fusion. I. Characterization of antibody families in primary and hyperimmune responses. Eur. J. Immunol. 130 (1978) 393-400.
MacLennan, I. C., Liu, Y. J., Johnson, G. D. Maturation and dispersal of B-cell clonal during T cell-dependent antibody responses. Immunol. Rev. 126 (1992) 143-161.
MacLennan, I. C. 1994. Germinal centers. Annu Rev Immunol 12:117-139.
Manz, R. A., A. Thiel, A. Radbruch. Lifetime of plasma cells in the bone marrow. Nature 388 (1997) 133-134.
Manz, R. A., A. E. Hauser, F. Hiepe, and A. Radbruch. 2005. Maintenance of serum antibody levels. Annu Rev Immunol 23:367-386.
Mascola, J. R., M. K. Louder, S. R. Surman, T. C. Vancott, X. F. Yu, J. Bradac, K. R. Porter, K. E. Nelson, M. Girard, J. G. McNeil, F. E. McCutchan, D. L. Birx, and D. S. Burke. 1996. Human immunodeficiency virus type 1 neutralizing antibody serotyping using serum pools and an infectivity reduction assay. AIDS Res Hum Retroviruses 12:1319-1328.
Mbawuike, I., C. L. Acuna, K. C. Walz, R. L. Atmar, S. B. Greenberg, and R. B. Couch. 1997. Cytokines and impaired CD8⁺ CTL activity among elderly persons and the enhancing effect of IL-12. Mech. Ageing Dev. 94:25-39.
Mbawuike, I. N., A. R. Lange, and R. B. Couch. 1993. Diminished influenza A virus-specific MHC class I-restricted cytotoxic T lymphocyte activity among elderly persons. Viral. Immunol. 6:55-64.
Mbawuike, I. N., C. Acuna, D. Caballero, K. Pham-Nguyen, B. Gilbert, P. Petribon, and M. Harmon. 1996. Reversal of age-related deficient influenza virus-specific CTL responses and IFN-gamma production by monophosphoryl lipid A. Cell. Immunol. 173:64-78.
Mbawuike, I. N., P. R. Wyde, and P. M. Anderson. 1990. Enhancement of the protective efficacy of inactivated influenza A virus vaccine in aged mice by IL-2 liposomes. Vaccine 8:347-352.
Mbawuike, I. N., S. Pacheco, C. L. Acuna, K. C. Switzer, Y. Zhang, and G. R. Harriman. 1999. Mucosal immunity to influenza without IgA: an IgA knockout mouse model. J. Immunol. 162:2530-2537.
Mbawuike, I. N., Y. Zhang, and R. B. Couch. 2007. Control of mucosal virus infection by influenza nucleoprotein-specific CD8⁺ cytotoxic T lymphocytes. Respir Res 8:44.
McMicael, A. J., F. M. Gotch, G. R. Noble, and P. A. Beare. 1983. Cytotoxic T cell immunity to influenza. N. Eng. J. Med. 309:13-17.
McHeyzer-Williams, M. G., M. J. McLean, P. A. Lalor, and G. J. Nossal. 1993. Antigen-driven B cell differentiation in vivo. J Exp Med 178:295-307.
Miller, C., Kelsoe, G. Ig V_{H} hypermutation is absent in the germinal centers of aged mice. J. Immunol. 155 (1995) 3377-3384.
Miller, R. A. 1995. Cellular and biochemical changes in the aging mouse immune system. Nutr. Rev. 53(Suppl 2):8-17.
Miller, R. A., Cellular and biochemical changes in the aging mouse immune system. Nutr. Rev. 53 (Suppl. 2) (1995) 8-10.
Miller, R. A. Aging and immune function. Int. Rev. Cytol. 124 (1991) 187-215.
Moulard, M., S. K. Phogat, Y. Shu, A. F. Labrijn, X. Xiao, J. M. Binley, M. Y. Zhang, I. A. Sidorov, C. C. Broder, J. Robinson, P. W. Parren, D. R. Burton, and D. S. Dimitrov. 2002. Broadly cross-reactive HIV-1-neutralizing human monoclonal Fab selected for binding to gp120-CD4-CCR5 complexes. Proc Natl Acad Sci U S A 99:6913-6918.
Mouton, C. P., O. Bazaldua, B. Pierce, and D. V. Espino. 2001. Common infections in older adults. Am. Fam. Physician 63:257-268.
Nichol, K. L., J. Wuorenma, and T. von Sternberg. 1998. Benefits of influenza vaccination for low-, intermediate-, and high-risk senior citizens. Arch Intern Med. 158:1769-1776.
Nie, X., S. Basu, J. Cemy. Immunization with immune complex alters the repertoire of antigen-reactive B cells in the germinal center. Eur. J. Immunol. 27 (1997) 3517-3525.
Nimmerjahn, F., and J. V. Ravetch. 2005. Divergent immunoglobulin G subclass activity through selective Fc receptor binding. Science 310:1510-1512.
Nimmerjahn, F., and J. V. Ravetch. 2006. Fcγ receptors: Old friends and new family members. Immunity 24:19-28.
Nimmerjahn, F., P. Bruhns, K. Horiuchi, and J. V. Ravetch. 2005. FcγRIV: a novel FcR with distinct IgG subclass specificity. Immunity 23:41-51.
Nossal, G. J. V., Abbot, A., Mitchell, J., Lummus, Z. Antigens in immunity. XV. Ultrastructural features of antigen capture in primary and secondary follicles. J. Exp. Med. 127 (1968) 277-290.
Oldstone, M. B. 1994. The role of cytotoxic T lymphocytes in infectious disease: history, criteria, and state of the art. Curr Top Microbiol Immunol 89:1-8.
Paul, S., S. Planque, Y. X. Zhou, H. Taguchi, G. Bhatia, S. Karle, C. Hanson, and Y. Nishiyama. 2003. Specific HIV gp120-cleaving antibodies induced by covalently reactive analog of gp120. J Biol Chem 278:20429-20435.
Paramithiotis, E., M. D. Cooper. Memory B lymphocytes migrate to bone marrow in humans. Proc. Natl. Acad. Sci. USA 94 (1997) 208-212.
Pawelec, G. 1999. Immunosenescence: impact in the young as well as the old? Mech. Ageing. Dev. 108:1-7.
Pearse, R. N., T. Kawabe, S. Bolland, R. Guinamard, T. Kurosaki, and J. V. Ravetch. 1999. SHIP recruitment attenuates Fcγ RIIB-induced B cell apoptosis. Immunity 10:753-760.
Pereira, M. S., P. Chakraverty, G. C. Schild, M. T. Coleman, and W. R. Dowdle. 1972. Prevalence of antibody to current influenza viruses and effect of vaccination on antibody response. Brit. Med. J. 4:701-703.
Phair, J., C. A. Kauffman, A. Bjornson, L. Adams, and C. Linnemann. 1978. Failure to respond to influenza vaccine in the aged: correlation with B-cell number and function. J. Lab. Clin. Med. 92:822-828.
Po, J. L., E. M. Gardner, F. Anaraki, P. D. Katsikis, and D. M. Murasko. 2002. Age-associated decrease in virus-specific CD8⁺ T lymphocytes during primary influenza infection. Mech. Ageing. Dev. 123:1167-1181.
Potter, C. W., and J. S. Oxford. 1979. Determinants of immunity to influenza infection in man. Br. Med. J. 35:69-75.
Powers, D. C. 1993. Influenza A virus-specific cytotoxic T lymphocyte activity declines with advancing age. J. Am. Geriatr. Soc. 41:1-5.
Powers, D. C., and R. B. Belshe. 1993. Effect of age on cytotoxic T lymphocyte memory as well as serum and local antibody responses elicited by inactivated influenza virus vaccine. J. Infect. Dis. 167:584-592.
Qin, D., J. Wu, K. A. Vora, J. V. Ravetch, A. K. Szakal, T. Manser, J. G. Tew. Fc gamma receptor IIB on follicular dendritic cells regulates the B cell recall response. J. Immunol. 164 (2000) 6268-6275.
Rafiq, K., A. Bergtold, R. Clynes. Immune complex-mediated antigen presentation induces tumor immunity. J. Clin. Invest. 110 (2002) 71-79.
Ravetch, J. V. and S. Bolland. 2001. IgG Fc receptors. Annu. Rev. Immunol. 19: 275-290.
Ravetch, J. V., R R. A. Clynes. Divergent roles for Fc receptors and complement in vivo. Annu. Rev. Immunol. 16 (1998) 421-432.
Regnault, A., Lankar, D., Lacabanne, V., Rodriguez, A., Thery C., Rescigno, M., Saito, T., Verbeek, S. Bonnerot, C., Ricciardi-Castagnoli, P., Amigorena, S. Fcγ receptor-mediated induction of dendritic cell maturation and major histocompatibility complex class I-restricted antigen presentation after immune complex internalization. J. Exp. Med. 189 (1999) 371-380.
Remington's Pharmaceutical Sciences, 18th Ed. Mack Printing Company, 1990.
Schuurhuis, D. H., A. Loan-Facsinay, B. Nagelkerken, J. J. van Schip, C. Sedlik, C. J. Melief, J. S. Verbeek, F. Ossendorp. Antigen-antibody immune complexes empower dendritic cells to efficiently prime specific CD8⁺ CTL responses in vivo. J. Immunol. 168 (2002) 2240-2246.
Slifka, M. K., M. Matloubian, R. Ahmed. Bone marrow is a major site of long-term antibody production after acute viral infection. J. Virol. 69 (1995) 1895-1902.
Smith, K. G., A. Light, G. J. V. Nossal, D. Tarlinton. The extent of an affinity maturation differs between the memory and antibody-forming cell compartments in the primary immune response. EMBO J. 16 (1997) 2996-3006.
Song, H., X. Nie, S. Basu, M. Singh, J. Cerny. Regulation of V_{H} gene repertoire and somatic hypermutation in germinal center B cells by passively administrated antibody. Immunology 98 (1999) 258-266.
Strassburg, M. A., S. Greenland, F. J. Sorvillo, L. E. Lieb, and L. A. Habel. 1986. Influenza in the elderly: report of an outbreak and review of vaccine effectiveness reports. Vaccine 4:38-44.
Szakal, A. K., Z. F. Kapasi, A. Masuda, J. G. Tew. Follicular dendritic cells in the alternative antigen transport pathway: microenvironment, cellular events, age and retrovirus related alterations. Semin. Immunol. 4 (1992) 257-265.
Szakal, A. K., J. K. Taylor, J. P. Smith, M. H. Kosco, G. F. Burton, J. G. Tew. Kinetics of germinal center development in lymph nodes of young and aging immune mice. Anat. Rec. 227 (1990) 475-485.
Takahashi, Y., D. M. Cerasoli, J. M. Dal Porto, M. Shimoda, R. Freund, W. Fang, D. G. Telander, E. N. Malvey, D. L. Mueller, T. W. Behrens, and G. Kelsoe. 1999. Relaxed negative selection in germinal centers and impaired affinity maturation in bcl-xL transgenic mice. J Exp Med 190:399-410.
Takai, T. 2002. Roles of Fc receptors in autoimmunity. Nat. Rev. Immunol. 2: 580-592.
Takai, T., M. Li, D. Sylvestre, R. Clynes, and J. V. Ravetch. 1994. FcRy chain deletion results in pleiotrophic effector cell defects. Cell 76: 519-529.
Takai, T., M. Ono, M. Hikida, H. Ohmori, and J. V. Ravetch. 1996. Augmented humoral and anaphylactic responses in FcγRII-deficient mice. Nature 379:346-349.
Taylor, P. M., D. C. Wraith, and B. A. Askonas. 1985. Control of immune interferon release by cytotoxic T-cell clones specific for influenza. Immunology. 54:607-614.
Tew, J. G., Phipps, R. P., Mandel, T. E. The maintenance and regulation of the humoral immune response: persisting antigen and the role of follicular antigen-binding dendritic cells as accessory cells. Immunol. Rev. 53 (1980) 175-201.
Thompson, W. W., D. S. Shay, E. Weintraub, L. Brammer, N. Cox, L. J. Anderson, and K. F. Fukuda. 2003. Mortality associated with influenza and respiratory syncytial virus in the United States. JAMA 289:179-186.
Toellner, K., Gulbranson-Judge, A., Taylor, D. R., Sze, D. M., I. C. MacLennan, I. C. Immunoglobulin switch transcript production in vivo related to the site and time of antigen-specific B cell activation. J. Exp. Med. 183 (1996) 2303-2312.
Tsoko, G. C., Lambris, J. D., Finkelman, F. D., Anatassiov, E. D., June, C. H. Monovalent ligands of complement receptor 2 inhibit whereas polyvalent ligands enhance anti-Ig induced human B cell intracytoplasmic free calcium concentration. J. Immunol. 144 (1990) 1640-1645.
Ulmer, J. B., J. J. Donnelly, S. E. Parker, G. E. Rhodes, P. L. Felgner, V. J. Dwarki, S. H. Gromkowski, R. R. Deck, C. M. DeWitt, and A. Friedman. 1993. Heterologous protection against influenza by injection of DNA encoding a viral protein. Science 259:1745-1749.
Weinberger, J. Z., M. I. Green, B. Benacerraf, M. E. Dorf. Hapten-specific T cell responses to 4-hydroxy-3-nitropheny acetyl. I. Genetic control of delayed-type hypersensitivity by V and I-A region genes. J. Exp. Med. 149 (1979) 1136-1148.
Wen, Y. M., D. Qu, S. H. Zhou. Antigen-antibody complex as therapeutic vaccine for viral hepatitis B. Int. Rev. Immunol. 18 (1999) 251-258.
Wick G, and B. Grubeck-Loebenstein. 1997. The aging immune system: primary and secondary alterations of immune reactivity in the elderly. Exp. Gerontol. 32:401-413.
Yang, X., Stedra, J., Cerny, J. Relative contribution of T and B cells to hypermutation and selection of the antibody repertoire in germinal centers of aged mice. J. Exp. Med. 183 (1996) 959-970.
Zhang, Y., C. L. Acuna, K. C. Switzer, L. Song, R. Sayers, and I. N. Mbawuike. 2000. Corrective effects of interleukin-12 on age-related deficiencies in IFN-γ production and IL-12Rβ2 expression in virus-specific CD8⁺ T cells. J. Interferon Cytokine. Res. 20:235-245.
Zhang, Y., C. L. Acuna, K. C. Switzer, L. Song, R. Sayers, and I. N. Mbawuike. 2000. Corrective effects of interleukin-12 on age-related deficiencies in IFN-γ production and IL-12Rβ2 expression in virus-specific CD8⁺ T cells. J Interferon Cytokine Res. 20:235-245.
Zhang, Y., Y. Wang, X. Gilmore, K. Xu, P. R. Wyde, and I. N. Mbawuike. 2002. An aged mouse model for RSV infection and diminished CD8(+) CTL responses. Exp Biol Med (Maywood) 227:133-140.
Zharhary, D. Segev, Y. Gershon, H. The affinity and spectrum of cross-reactivity of antibody production in senescent mice: the IgM response. Mech. Ageing Dev. 6 (1977) 385392.
Zheng, B., S. Han, G. Kelsoe. T helper cells in murine germinal centers are antigen-specific emigrates that downregulate Thy-1. J. Exp. Med. 184 (1996) 1083-1091.
Zheng, B., Han, S., Takahashi, Y., Kelsoe, G. Immunosenescence and germinal center reaction. Immunol. Rev. 160 (1997) 63-77.
Zheng, B. J., M. H. Ng, L. F. He, X. Yao, K. W. Chan, K. Y. Yuen, Y. M. Wen. Therapeutic efficacy of hepatitis B surface antigen-antibodies-recombinant DNA composite in HBsAg transgenic mice. Vaccine 19 (2001) 4219-4225.
Zheng. B., Z. Z. Ozen, S. Cao, Y. Zhang, and S. Han. 2002. CD4-deficient T helper cells are capable of supporting somatic hypermutation and affinity maturation of germinal center B cells. Eur J. Immunol. 32:3315-3325.
Zheng, B., K. Switzer, E. Marinova, D. Wansley, and S. Han. 2007. Correction of age-associated deficiency in germinal center response by immunization with immune complexes. Clin Immunol 124:131-137.
Zheng, B., Y. Zhang, H. He, E. Marinova, K. Switzer, D. Wansley, I. Mbawuike, and S. Han. 2007. Rectification of age-associated deficiency in cytotoxic T cell response to influenza A virus by immunization with immune complexes. J Immunol 179:6153-6159.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of improving an immune response in an individual, comprising delivering to the individual a therapeutically effective amount of an immune complex, said complex comprising:
1) an antigen; and
2) an antibody that immunologically recognizes the antigen.

2. The method of claim 1, wherein the antigen is selected from the group consisting of a viral antigen, a bacterial antigen, and a fungal antigen.

3. The method of claim 2, wherein the viral antigen comprises an inactivated or attenuated intact viral particle.

4. The method of claim 2, wherein the viral antigen comprises at least part of a protein of the virus.

5. The method of claim 2, wherein the viral antigen is from Influenza, HIV, Hepatitis, SARS, or Varicella zoster virus.

6. The method of claim 5, wherein the viral antigen is from HIV.

7. The method of claim 5, wherein the viral antigen is from Influenza.

8. The method of claim 2, wherein the bacterial antigen comprises killed or attenuated whole bacteria.

9. The method of claim 2, wherein the bacterial antigen comprises at least part of a protein of a bacteria.

10. The method of claim 2, wherein the bacterial antigen is from Staphylococcus, Haemophilus, Streptococcus, Escherichia, Salmonella, Shigella, Yersinia, Klebsiella, Pseudomonas, Enterobacter, Salmonella, Serratia, or Proteus.

11. The method of claim 1, wherein the individual is elderly, very young, has an infection, is being treated for cancer, or has had an organ or tissue transplant.

12. The method of claim 1, wherein the method also comprises administration of an immunostimulatory agent to the individual.

13. The method of claim 1, wherein the antibody comprises IgG or IgM.

14. The method of claim 2, wherein the antigen comprises at least part of a fungal protein.

15. The method of claim 2, wherein the fungal antigen is from Candida, Aspergillus, Cryptococcus, Coccidioides, Histoplasma, Pneumocystis, or Paracoccidioides.

16. The method of claim 1, wherein the individual is an immune-compromised individual.

17. A kit for an immune-compromised individual, comprising an immune complex, said kit housed in a suitable container and comprising:
1) an antigen; and
2) an antibody that immunologically recognizes the antigen.

18. The kit of claim 17, wherein the antigen is selected from the group consisting of a bacterial antigen, fungal antigen, or viral antigen.
